(19) United States
(12) Patent Application Publication
(10) Pub. No.: US 2026/0000794 A1
(43) Pub. Date: Jan. 1, 2026

(54) IN VIVO TRANSITION METAL DETECTION

(71) Applicant: The University of Sydney, Sydney (AU)

(72) Inventors: Elizabeth NEW, Sydney (AU); Kay DOUBLE, Sydney (AU); Marcus GRAZIOTTO, Sydney (AU); Liam ADAIR, Sydney (AU)

(21) Appl. No.: 18/844,576

(22) PCT Filed: Mar. 8, 2023
(86) PCT No.: PCT/AU2023/050157

§ 371 Date: Sep. 6, 2024

(30) Foreign Application Priority Data
Mar. 8, 2022 (AU) 2022900560

(51) Int. Cl. A61K 51/04; A61B 5/00; A61B 6/03; A61B 6/50; A61K 49/00

(52) U.S. Cl. CPC A61K 51/0482; A61B 5/0071; A61B 6/037; A61B 6/501; A61K 49/0021; A61K 49/0052; A61K 2123/00

(57) ABSTRACT

The present invention is directed to a probe for detecting a transition metal, the probe comprising: a metal chelating portion configured to coordinate with a transition metal; a label portion configured to be detectable; and a linker bound to both the metal chelating portion and the label portion, wherein the linker is configured to react with a nucleophile when the metal chelating portion coordinates with the transition metal, as well in vivo and in vitro methods and uses thereof.

### CROSS REFERENCE TO RELATED APPLICATION

This application claims priority from Australian Provisional Patent Application No. 2022900560, filed on 8 Mar. 2022, the entire contents of which are incorporated herein.

### FIELD OF THE INVENTION

The present invention relates to the field of biochemistry. In particular, the present invention is directed to a probe for measuring the concentration and location of transition metals in vivo. However, it will be appreciated that the invention is not limited to this particular field of use.

### BACKGROUND

The following discussion of the prior art is provided to place the invention in an appropriate technical context and enable the advantages of it to be more fully understood. It should be appreciated, however, that any discussion of the prior art throughout the specification should not be considered as an express or implied admission that such prior art is widely known or forms part of the common general knowledge in the field.

Transition metals, such as copper, zinc, iron, cobalt and the like are vital trace elements required for many biological processes. For instance, it is known that copper is critical for maintaining brain health and the functioning of neurons, zinc can have a role in protein folding (in maintaining structural motifs known as ‘zinc fingers’), and both copper and zinc are found in enzymes, such as superoxide dismutase (i.e., CuZnSOD).

However, when the biologically available levels of these transition metals deviate from normal levels (i.e., metal dyshomeostasis), this can manifest as a number of disease states or conditions in a subject. For instance, in a subject that has a significant systemic copper deficiency, such as that which occurs in Menkes disease, the affected individual can suffer from a range of symptoms, including neurological effects such as seizures, intellectual disability and an unstable body temperature. On the other hand, copper is also potentially toxic in high concentrations, and in a subject that has a significant systemic copper excess, such as that seen in Wilson's disease, organs of the body such as the brain and liver suffer oxidative damage, leading to symptoms ranging from jaundice and loss of appetite, to neurological symptoms such as hand tremors, difficulty with movement and/or speech, personality changes, confusion, dementia and depression.

Further, some conditions are characterised by a change in transition metal levels in affected tissues, such as a regional copper deficiency in affected tissues in amyotrophic lateral sclerosis (ALS; also known as Lou Gehrig's disease) and Parkinson's disease (PD), and accumulation of copper in neurofibrillary tangles and plaques in Alzheimer's disease (AD). Tumours are also known to accumulate copper to drive rapid growth, therefore locally high copper levels may be able useful in identifying potential fast growing tumours.

Additionally, there are currently chemotherapy agents being developed that are based on transition metals, such as copper, for the treatment of cancer, including cancers of the central nervous system. Such emerging agents utilise the oxidative toxicity of copper to ameliorate cancer cells.

However, an issue that arises with developing or monitoring treatments directed to ameliorating metal dyshomeostasis or delivering transition metals to the CNS is that there is currently no method available for directly quantifying transition metals in the CNS of living subjects, or mapping the distribution of transition metal throughout the brain and spinal cord. Current methods require the analysis of post mortem samples, or the indirect measurement of transition metals via the imaging of structures in living subjects thought to be associated with transition metal dyshomeostasis, such as the plaques associated with AD, or via samples taken systemically (such as blood samples). However, there are no current reliable methods for detecting transition metals in particular regions or tissues of a living subject.

Accordingly, there is a need for a method that is capable of measuring transition metal quantity and/or location in the CNS of a living subject. There is also a need for a compound that is capable of interacting with transition metals in the CNS in vivo and is also detectable with known imaging techniques.

It is an object of the present invention that at least one of the needs above is at least partially satisfied.

It is an object of the present invention to overcome or ameliorate one or more the disadvantages of the prior art, or at least to provide a useful alternative.

### SUMMARY OF THE INVENTION

The present invention aims to address at least one of the deficiencies in the monitoring of, and/or measurement of, transition metal concentration and/or location in a living subject. In particular, the present invention aims to provide a probe that is capable of, or adapted to, measure the location and/or relative density of particular transition metal(s) in a subject, by utilising widely used medical imaging techniques and/or common analytical techniques, as well as methods for the use of such probes.

In one aspect of the present invention, there is provided a probe for detecting a transition metal, the probe comprising: a metal chelating portion configured to coordinate with a transition metal; a label portion configured to be detectable; and a linker bound to both the metal chelating portion and the label portion, wherein the linker is configured to react with a nucleophile when the metal chelating portion coordinates with the transition metal.

The transition metal detected by the probe of the present invention may be selected from Cu(I), Cu(II), Zn(II), Fe(II), Fe(III), Co(II), Mn(II), Ni(II) or Cd(II) or any other suitable transition metal species capable of forming at least one coordination bond. In one embodiment, it may be Cu(I). The transition metal may be detected in a biological tissue. It may be detected in vivo and/or in an ex vivo sample.

The metal chelating portion of the probe of the present invention may comprise at least one nitrogen and/or sulfur and/or oxygen heteroatom which coordinates to the transition metal. It would be appreciated by the skilled person that a lone pair of electrons on the heteroatom(s) forms a covalent coordination bond with the transition metal. In some embodiments, the metal chelating portion may comprise at least one, or at least two, sulfur heteroatoms. The sulfur heteroatom may be provided by an organic sulfide group of general formula R—S—R′, whereby R and R′ may each be any suitable carbon-containing moiety.

In some embodiments, the metal chelating portion is of general formula (I):

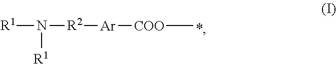

wherein:
Ar is an aromatic heterocycle group comprising at least one nitrogen heteroatom, optionally further comprising a sulfur heteroatom;
each R¹ is independently an organic group of general formula R³—S—R^{3′}, R³—O—R^{3′}, or R³—N—R^{3′}
each R², R³ and R^{3′} are independently straight chained or branched C₁-C₆-alkyl; and
* is bound to the linker.

The Ar group may be selected from the group consisting of pyrrolinyl, pyrrolyl, pyrazolinyl, imidazolinyl, imidazolyl, triazolyl, tetrazolyl, isothiazolyl, thiazolyl, thiadiazolyl, pyridinyl, pyridazinyl, pyrimidinyl, pyrazinyl, triazinyl and thiazinyl or any other suitable aromatic heterocycle group comprising at least one nitrogen heteroatom.

In some embodiments, the metal chelating portion is

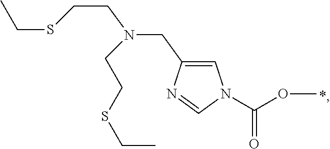 [SMILES: CCSCCN(CCSCC)Cc1cn(C(=O)OC)cn1]

which corresponds to a compound of general formula (I) whereby Ar is imidazolyl, R² is methyl, both R¹ groups are an organic sulfide group of general formula R³—S—R^{3′}, and all R³ and R^{3′} groups are ethyl.

The label portion of the probe of the present invention may comprise a label. The label may be detectable by at least one of fluorescence, PET imaging, MRI imaging or CT imaging. The label may be detectable by both fluorescence and PET imaging. Accordingly, the label portion may comprise a fluorophore or a luminescent marker and/or a radiolabel. The skilled person would appreciate that a fluorophore is detectable by fluorescence and a radiolabel is detectable by PET imaging. The fluorophore may be any suitable fluorophore. In some embodiments, it may be a naphthalimide fluorophore. It may be naphthalimide. It may be 4-amino-1,8-naphthalimide or a substituted derivative thereof. It may be

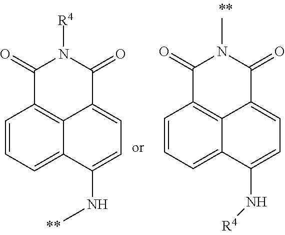

wherein: ** is bound to the linker; and R⁴ is C₁-C₆ alkyl, which may be further substituted with one or more substituents selected from amino, carboxyl, C₃-C₆ aryl and halo, wherein the aryl may be further substituted with one or more substituents selected from amino, carboxyl, C₁-C₆ alkyl, C₃-C₆ aryl and halo. Each alkyl may contain 1, 2 or 3 heteroatoms each selected from a nitrogen atom, an oxygen atom and a sulfur atom. R⁴ may optionally comprising the radiolabel. The radiolabel may be selected from the group consisting of ¹¹C, ¹³N, ¹⁵O, ¹⁸F and ¹³¹I. In some embodiments, it may be ¹⁸F.

The probe of the present invention also comprises a linker that is covalently bound to both the metal chelating portion and the label portion. Preferably, the linker portion separates the metal chelating portion and the label portion by being positioned between these two portions. In other words, it is preferred that the metal chelating portion is bound at one end of the linker and the label portion is bound at the other end of the linker (i.e., [metal chelating portion]-(linker)-[label portion]), although alternative arrangements may be used. In some embodiments, the linker may be C₁-C₆ alkyl, whereby the alkyl may be straight chained or branched C₁-C₆ alkyl and may further comprise 1, 2 or 3 heteroatoms selected from a nitrogen atom, an oxygen atom and a sulfur atom. In one embodiment, the linker may be —CH₂—CH₂—O—CH₂—CH₂—. In another embodiment, the linker may be —CH₂—CH₂—.

The probe of the present invention may be adapted, or designed, so as to be able to access specific parts of the subject, and/or accumulate in specific organs or regions of the subject. For instance, the probe of the present invention may be adapted to cross the blood-brain barrier in a subject, so as to allow detection of a transition metal in the central nervous system of the subject. The skilled person would understand that compounds that cross the blood brain barrier by diffusion are generally lipophilic, relatively small (i.e., up to about 600 Da), and possess a positive charge at physiological pH. In one embodiment, the probe of the present invention that is capable of crossing the blood brain barrier is selected from the compounds:

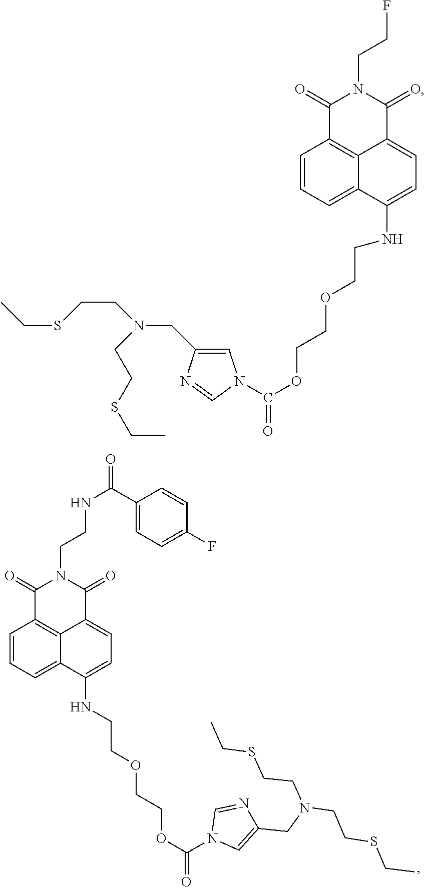

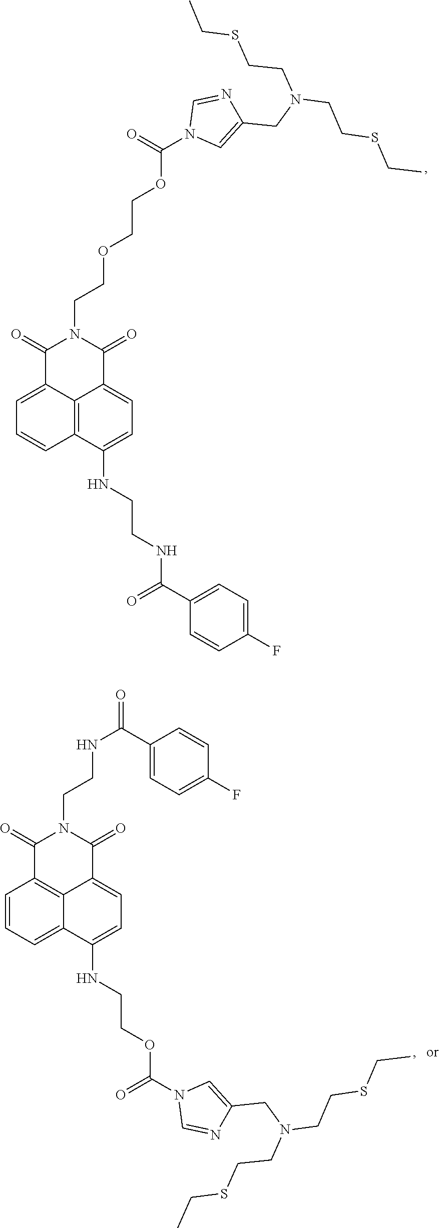 [SMILES: CCSCCN(CCSCC)Cc1cn(C(=O)OCCOCCN2C(=O)c3cccc4c(NCCNC(=O)c5ccc(F)cc5)ccc(c34)C2=O)cn1 ; CCSCCN(CCSCC)Cc1cn(C(=O)OCCNc2ccc3c4c(cccc24)C(=O)N(CCNC(=O)c2ccc(F)cc2)C3=O)cn1]

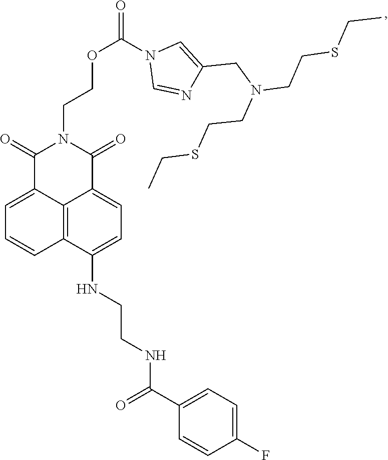 [SMILES: CCSCCN(CCSCC)Cc1cn(C(=O)OCCN2C(=O)c3cccc4c(NCCNC(=O)c5ccc(F)cc5)ccc(c34)C2=O)cn1]

wherein the F may be ¹⁸F (i.e., a radiolabel) or ¹⁹F.

In one embodiment, the compound is:

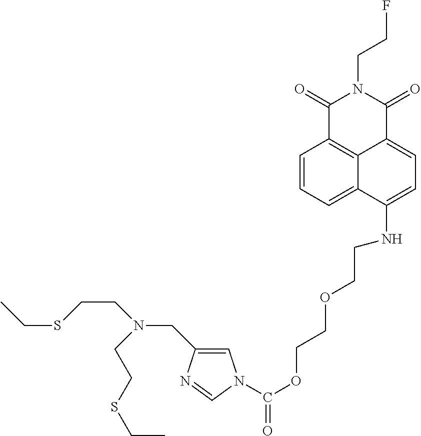 [SMILES: CCSCCN(CCSCC)Cc1cn(C(=O)OCCOCCNc2ccc3c4c(cccc24)C(=O)N(CCF)C3=O)cn1]

wherein the F may be ¹⁸F (i.e., a radiolabel) or ¹⁹F.

Without being bound to theory, it is understood that use of the probe of the present invention includes the transition metal coordinating with the metal chelating portion to form an activated probe, and the activated probe then reacting with a nucleophile, to form an immobilised conjugate comprising the nucleophile, the linker and the label portion. In one embodiment, the nucleophile is a biological structure found in a biological tissue, such as for example, an amino acid sidechain that is present in a protein or a peptide. When the probe of the present invention is immobilised to a biological structure and the label portion detected, this is equivalent to the detection of a transition metal in a biological tissue.

In a second aspect of the present invention, there is provided a method for measuring a transition metal in vivo, comprising administering to a subject an effective amount of the probe described herein and detecting the label portion. The measuring may comprise locating the label portion, and/or detecting regions of high or low intensity of the label portion. For instance, a plurality of label portions may be detected in a particular region or area, which leads to the identification of, or measurement of, regions of relatively high intensity of label portions being detected, from which a relatively high concentration of transition metal can be inferred.

In a third aspect of the present invention, there is provided a method of identifying a region of transition metal dyshomeostasis in a subject, comprising administering to a subject an effective amount of the probe described herein, locating the label portion and comparing the location of the label portion to a control subject without transition metal dyshomeostasis.

The subject may be a mammal. The subject may be a human or it may be an animal (i.e., the probe of the present invention may be suitable for use in medicine or in veterinary medicine). The animal may be a companion animal (such as a cat or a dog), or it may be a livestock animal (such as a cow, a sheep, a goat, a pig, or poultry) or it may be any other mammal. In one embodiment, the subject may be a human. In another embodiment, the subject may be a dog.

The region of transition metal dyshomeostasis in the subject may be located in any particular system, organ, tissue, cell, or any combination thereof, that may be affected by transition metal dyshomeostasis. The region may comprise a region of the central nervous system, or the endocrine system, or the gastrointestinal system, or the hepatic system, or the renal system, or any other suitable biological system. In one embodiment, it may be a region of the central nervous system. The region of the central nervous system may be the brain, or a localised area of the brain. When the region of transition metal dyshomeostasis is in the central nervous system, and more particularly the brain, of the subject, this may be indicative of a neurological condition in the subject. The neurological condition may be selected from stroke, Parkinson's disease, Alzheimer's disease, amyotrophic lateral sclerosis (ALS), Wilson's disease, multiple sclerosis (MS), and Menkes disease or it may be a cancer, such as neuroblastoma. Similar conclusions may be formed when the region of transition metal dyshomeostasis is in another biological system; for example, the region of transition metal dyshomeostasis may be a region of the endocrine system, more particularly the pancreas, of the subject, which may be indicative of the subject experiencing, or suffering from, diabetes mellitus. In another example, the region of transition metal dyshomeostasis in the subject may be the liver, which may be indicative of Wilson's disease in a human (when considered alone or in combination with detection of the label portion in the central nervous system of the subject), or copper storage hepatopathy in a dog.

In a fourth aspect of the present invention, there is provided a method of diagnosing a neurological condition in a subject, comprising the steps of: administering to the subject an effective amount of the probe described herein and detecting the label portion in the central nervous system of the subject to form a subject map of label intensity; administering to a control subject an effective amount of the probe described herein and detecting the label portion in the central nervous system of the control subject to form a control map of label intensity; and comparing the subject map of label intensity to a control map of label intensity; whereby a region of relative low intensity or high intensity in the subject map of label intensity is indicative of a neurological condition, wherein the neurological condition selected from stroke, Parkinson's disease, Alzheimer's disease, amyotrophic lateral sclerosis (ALS), Wilson's disease, multiple sclerosis (MS), Menkes disease, and a neurological cancer, such as neuroblastoma.

In the methods described herein, there may be a further step, comprising a period of time between administering the probe to the subject or the control subject and locating the label portion. As the skilled person would appreciate from the present disclosure, the advantage of the present invention is the immobilisation of the label portion at the site of transition metal detection or recognition. Accordingly, a period of time between administration of the probe to the subject and the detection of the label portion may be required in order to provide sufficient time for the probe to, for instance, diffuse to, and/or accumulate in, the region(s) of interest, and either recognise a transition metal and react with a nucleophile to immobilise the label portion, or otherwise be eliminated from that region so as to not provide a background signal of mobile label portion. The period of time may be any suitable amount of time. It may be between about 1 minute and about 2 hours, or between about 3 minutes and about 1 hour, or between about 5 minutes and about 30 minutes, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119 or 120 minutes, or any range therein. In one embodiment, the period of time is between about 3 minutes and about 1 hour. The skilled person would appreciate that factors such as administration route, biological region of interest and radiolabel half-life may impact the period time length.

The probe of the present invention, when used to measure or detect a transition metal in vivo, is administered to a subject, and/or a control subject where applicable. The administering may be by any suitable route. For example, the administering may be selected from oral administration, intranasal administration, subcutaneous injection or infusion and intravenous injection or infusion. In one embodiment the administering is by intravenous infusion.

In a fifth aspect of the present invention, there is provided a method of labelling a site of a transition metal in a biological tissue, comprising:
(a) contacting the probe described herein with the biological tissue, wherein the metal chelating portion of the probe binds to the transition metal, whereby binding of the transition metal to the metal chelating portion cleaves the metal chelating portion from the linker to form a reactive acyl group;
(b) contacting the reactive acyl group with a nucleophile, resulting in binding of the label portion and the linker to the nucleophile; and
(c) measuring the location of the label portion.

The method of the fifth aspect may include labelling a biological tissue in vivo or it may include labelling an ex vivo biological tissue sample. In other words, the probe of the present invention may be used as both an administered imaging agent for in vivo use, or it may be used as a laboratory reagent for investigating transition metal distribution in tissue samples taken from a subject.

In each of the aforementioned methods, the nucleophile may be any suitable nucleophile. It may be an amino acid or an amino acid with a nucleophile side chain. The nucleophile may be a free thiol group or it may be a free amino group.

The skilled person would be aware that the above aspects and associated options may be used alone or together in any suitable combination.

### BRIEF DESCRIPTION OF DRAWINGS

This patent application contains at least one drawing executed in color. Copies of this patent application with color drawing(s) will be provided by the Office upon request and payment of the necessary fees.

Embodiments of the present invention will now be described, by way of example only, with reference to the accompanying figures.

FIG. 1: Absorbance of 10 μM F—NpCul (compound of Formula (IV)) in PBS buffer, at pH=7.4.

FIG. 2: Excitation and emission spectra of 10 μM F—NpCul (compound of Formula (IV)) in PBS buffer, at pH=7.4.

FIG. 3: Quantifying cortical Cu levels in the intact mouse brain with the Deep In Vivo Explorer (DIVE) multiphoton microscope. Intravenous injection of a fluorescent dextran tracer (1) enables vascular mapping (red; 4) in the parietal cortex (2). Subsequent retro-orbital injection of F—NpCul enabled real time 3D imaging of Cu levels via Cu-recognition and accumulation of the Cu-activated F—NpCul-protein complex (3) in the brain tissue (green; 5) demonstrating F—NpCul is readily BBB permeable.

FIG. 4: Mean fluorescence intensities of F—NpCul (10 μM) in SH-SY5Y cells treated with a vehicle control, 10 μM CuATSM (copper-loading agent) or 100 μM BCS (copper-depleting agent) for 1 h. Mean intensity of 40-50 regions of interest from at least 10 images.

FIG. 5: Fluorescentmission quantified longitudinally from real-time fluorescent images of anaesthetised SWISS mice obtained within the Deep In vivo Explorer (DIVE) microscope following F—NpCul injection.

FIG. 6: Representative fluorescent images from one control (untreated) and one CuATSM-treated (copper treatment) anaesthetised mouse injected with 4 mg/kg F—NpCul and imaged with the DIVE microscope.

### DETAILED DESCRIPTION

### I. Definitions

The following definitions are provided to enable the skilled person to better understand the invention disclosed herein. These are intended to be general and are not intended to limit the scope of the invention to these terms or definitions alone. Unless defined otherwise, all technical and scientific terms used herein have the same meaning as commonly understood by one having ordinary skill in the art to which the invention pertains.

The term “alkyl” as used herein refers to a hydrocarbon radical derived from an alkane, which may be linear, branched or cyclised. For instance, “methyl” refers to a radical group derived from methane.

The term “alkenyl” as used herein refers to a hydrocarbon radical derived from an alkene, which may be linear, branched or cyclised. For instance, “ethenyl” refers to a radical group derived from ethene.

The term “aryl” as used herein refers to a hydrocarbon radical derived from an aromatic hydrocarbon. For instance, “phenyl” refers to a radical group derived by a benzene ring.

The term “nucleophile” as used herein refers to any chemical species that forms bonds with electrophiles by donating an electron pair. As the skilled person would appreciate, all molecules or ions with a free pair of electrons or at least one pi bond can act as a nucleophile. Likewise, “electrophile” as used herein refers to a chemical species that forms bonds with a nucleophile by accepting an electron pair.

As used herein, the term “comprising” means “including”. Variations of the word “comprising”, such as “comprise” and “comprises”, have correspondingly varied meanings. As used herein, the terms “including” and “comprising” are non-exclusive. As used herein, the terms “including” and “comprising” do not imply that the specified integer(s) represent a major part of the whole.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition, process or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising”, it should be readily understood that (unless otherwise stated) the description should be interpreted to also describe such an invention using the terms “consisting essentially of” or “consisting of.” In other words, with respect to the terms “comprising”, “consisting of”, and “consisting essentially of”, where one of these three terms are used herein, the presently disclosed and claimed subject matter may include the use of either of the other two terms. Thus, in some embodiments not otherwise explicitly recited, any instance of “comprising” may be replaced by “consisting of” or, alternatively, by “consisting essentially of”.

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be non-restrictive regarding the number of instances (i.e., occurrences) of the element or component. Therefore “a” or “an” should be read to include one or at least one, and the singular word form of the element or component also includes the plural unless the number is obviously meant to be singular.

Other than in the operating examples, or where otherwise indicated, all numbers expressing quantities of ingredients or reaction conditions used herein are to be understood as modified in all instances by the term “about”. The examples are not intended to limit the scope of the invention. In what follows, or where otherwise indicated, “%” will mean “weight %”, “ratio” will mean “weight ratio” and “parts” will mean “weight parts”.

The terms “predominantly” and “substantially” as used herein shall mean comprising more than 50% by weight, unless otherwise indicated.

As used herein, with reference to numbers in a range of numerals, the terms “about”, “approximately” and “substantially” are understood to refer to the range of −10% to +10% of the referenced number, preferably −5% to +5% of the referenced number, more preferably −1% to +1% of the referenced number, most preferably −0.1% to +0.1% of the referenced number. Moreover, with reference to numerical ranges, these terms should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, from 8 to 10, and so forth.

As used herein, wt. % refers to the weight of a particular component relative to total weight of the referenced composition.

The complete disclosures of the patents, patent documents and publications cited herein are incorporated by reference in their entirety as if each were individually incorporated.

The following description conveys exemplary embodiments of the present invention in sufficient detail to enable those of ordinary skill in the art to practice the present invention. Features or limitations of the various embodiments described do not necessarily limit other embodiments of the present invention or the present invention as a whole. Hence, the following detailed description does not limit the scope of the present invention, which is defined only by the claims.

The present invention relates to a probe for measuring the quantity and/or location of transition metals in biological tissue, as well as methods for the use of the probe.

In particular, the inventors have developed a probe for the measurement of, or detection of, transition metals in either in vivo or ex vivo/post mortem tissue. The probe is a modular probe that is advantageously detectable using known medical imaging techniques and/or common analytical techniques. As will be described in more detail below and with reference to the examples, the probe of the present invention has been advantageously designed so that the portion of the probe detectable by imaging techniques (herein referred to as the “label portion”) is immobilised at the site that the transition metal was recognised by the probe, allowing for a larger window of time in which the imaging can occur without diffusion of the bound probe into surrounding tissues or elimination of the complexed transition metal and hence a more accurate representation of the distribution of transition metals in the tissues. A further advantage is that, unlike prior art probes, the label portion does not require binding directly to the transition metal in order to be detectable. Additionally, it is also an advantage that the probe provides a non-invasive method for measuring transition metal concentrations and locations in a living subject. In other words, the probe of the present invention advantageously provides the ability to use transition metals as new biomarkers for detecting and diagnosing conditions, and/or for monitoring the effect of medications that affect transition metal levels, and/or for monitoring disease progression independent of treatment, or as a research tool for studying the natural biology of transition metals and their interactions in mammalian systems (i.e., studying normal distribution, movement and binding or interaction patterns of transition metals in healthy mammals).

It is envisioned by the inventors that the probe may be used for diagnostic purposes (i.e., measuring and identifying regions of relative high or relative low concentrations of a transition metal that may be indicative of a dyshomeostasis disorder, or for detecting heavy metal pollutants or toxicants in a subject) or for quantifying or measuring treatments that comprise the transition metal or affect the level of the transition metal (i.e., identifying changes in transition metal concentrations after administration of a compound comprising the transition metal, or a chelator for binding to and eliminating the transition metal) or as an index of disease progression in a disorder that is characterised by transition metal dyshomeostasis (i.e., either the progressive accumulation, or deficiency, of transition metals).

### II. Probe

Compounds that bind to, or are accumulated in, particular structures, tissues or components of biological systems and which are detectable by common imaging or analytical techniques are known. Such compounds may also be referred to as probes, contrast agents, imaging agents or similar terms of the art. Generally, such compounds generally comprise a portion that binds to a certain structure or accumulates in a certain tissue, and a label that is detectable by standard medical imaging or analytical techniques. For example, positron emission tomography (PET) scanning detects radioactive atoms that undergo beta plus (β⁺) decay (i.e., positron emitters) that can be used to radiolabel compounds, which are then detected by the PET scanners. An example of a PET contrast agent is fluorodeoxyglucose (FDG) which is essentially a glucose molecule labelled with ¹⁸F, a positron emitter. When administered to a subject, the FDG is taken up and accumulates in tissues with a high energy need, such as active tumours.

Whilst most prior art probes or contrast agents accumulate in tissues (such as FDG) or bind to certain proteins or motifs, some have been developed for generally visualising or imaging the location and/or quantity of transition metals in tissues, including cells. However, known compounds for visualising transition metals rely on fluorescence, whereby a non-detectable, ‘off’ compound is ‘switched on’ by binding to a particular transition metal, leading the ‘on’ complex (i.e., the ‘off’ probe and the transition metal) to fluoresce and detection of the transition metal to occur. However, as the skilled person will appreciate, fluorescence relies on generally visible light to excite the fluorophore and then emit a different wavelength, and in particular the use of an excitation wavelength to illuminate the complex and an emission wavelength to be detected, which is only useful for analysing tissue or cell samples studied in vitro or taken from a subject, such as by biopsy or post mortem sampling, that can be directly illuminated. As would be clear to the skilled person, fluorescent probes are not suitable for use in a living subject, particularly not for measuring transition metal location or concentration within organs of a living subject.

However, the probe of the present invention has advantageously been developed to recognise a transition metal in situ and immobilise the label portion at the location that the transition metal was identified, whereby the label portion is adapted to be able to be detected from outside the body of a living subject (i.e., does not rely solely on fluorescence for detection).

Accordingly, the probe of the present invention comprises a metal chelating portion, a label portion, and a linker located between the metal chelating portion and the label portion. The probe is adapted for detecting a transition metal in a biological tissue. The probe is of a modular design, whereby the metal chelating portion, the linker and the label portion are independently adaptable for their respective uses and no one portion relies on an interaction with any other portion in order to be detectable. In other words, the label portion does not require binding to or with a transition metal in order to be detectable, contrary to known contrast agents used for identifying transition metals. The probe may also be adapted to be able to cross the blood brain barrier, to allow for the detection of transition metal(s) in the central nervous system, or the probe may be adapted to accumulate in another organ or compartment, such as the liver, pancreas or kidneys. As the skilled person will appreciate, in order to cross the blood brain barrier by diffusion (as opposed to active transport), a compound such as a probe must be lipophilic, relatively small (i.e., between about 400 and 600 Da, or no more than 600 Da), and possess a positive charge at physiological pH. The probe may be suitable to broadly detect transition metals concentrations throughout the central nervous system, or it may be suitable to identify local regions of high and/or low concentrations of transition metals. The probe may be suitable for detecting transition metals in an oxidising environment or in a reducing environment, for example the probe may be suitable for detecting transition metals within cells or in a redox-active pathological extracellular structure such as an amyloid plaque deposit or neurofibrillary tangle.

### A. Transition Metal

The probe of the present invention may be adapted to detect any transition metal. By “transition metal”, it is meant “an element whose atom has a partially filed d sub-shell, or which can give rise to cations with an incomplete d sub-shell”. In other words, the transition metal of the present invention is any element in the d-block of the periodic table (i.e., between group 4 and group 11 of the periodic table). The transition metal being detected by the probe may be an essential biological element that is required for at least one biological process, or it may be an element that is not essential for a biological process. The transition metal may be a pollutant or a toxicant. The transition metal may be an essential trace element. The transition metal may be, for example, copper (Cu), zinc (Zn), iron (Fe), cobalt (Co), manganese (Mn), nickel (Ni), cadmium (Cd), chromium (Cr), molybdenum (Mo), mercury (Hg), or vanadium (V). As the skilled person will appreciate, transition metals may be found in different oxidation states. For example, the transition metal being detected by the probe may be Cu (I), Cu (II), Zn (II), Fe (II), Fe (III), Co (II), Mn (II), Ni (II), Cd (II), Cr (II), Cr (III), Cr (VI), Mo (II), Mo (III), Hg (I), Hg (II) or V (II). In one embodiment, the transition metal is selected from Cu (I), Cu (II), Zn (II), Fe (II), Fe (III), Co (II), Mn (II), Ni (II) or Cd (II). For example, the transition metal is Cu (I).

### B. Metal Chelating Portion

The metal chelating portion of the present invention is adapted to coordinate with a transition metal. As the skilled person would appreciate, a coordinate bond, also referred to as a dative covalent bond or a dipolar bond, is a covalent bond that forms between a metal ion and a heteroatom with a lone pair, such as a nitrogen atom or an oxygen atom or a sulfur atom, whereby a bond is formed when the lone pair of the heteroatom is donated to the metal ion. In other words, the coordinate bond forms when one atom, usually N, S or O, donates two electrons to a metal ion to form a covalent bond.

The metal chelating portion of the present invention is also advantageously adapted to react with a nucleophile when it coordinates with, or forms at least one coordination bond with, a transition metal. Put broadly, in use, the probe of the present invention undergoes a first phase when the metal chelating portion complexes with a transition metal (referred to as a ‘recognition’ phase) to produce an ‘activated probe’, and a second step whereby a nucleophile reacts with the activated probe, cleaving the metal chelating portion from the linker and immobilising the linker and the label portion (referred to as a ‘cleavage’ or ‘immobilising’ phase). Without being bound to theory, it is understood by the inventors that the transition metal, when complexed with the metal chelating portion, results in electron density being drawn away from the nitrogen heteroatom of the aromatic heterocycle group of the metal chelating portion, therefore weakening the bond between the aromatic heterocycle group and the carboxyl group, reducing electron density on the carboxyl carbon of the metal chelating portion, turning this carboxyl carbon into an electrophile. This allows for a nucleophile, which donates an electron pair to an electrophile, to form a new covalent bond between the nucleophile-containing moiety and the carboxyl group, whilst simultaneously cleaving the metal chelating portion transition metal complex from the linker. The nucleophile may be any suitable nucleophile. In some embodiments, the nucleophile may be an amino acid, or a sidechain of an amino acid, of a protein or peptide located near to the transition metal. The nucleophile may be a Lewis base. The amino acid may be arginine, lysine, histidine, cysteine, aspartic acid, glutamic acid, tyrosine, which are known to comprise sidechains that are nucleophilic when neutral. The nucleophile may be an anion, or an anionic site on a macromolecule such as a protein or a nucleic acid chain. The nucleophile may be thiol group or an amine group present in a protein or peptide.

In one embodiment, the metal chelating portion is of general formula (I):

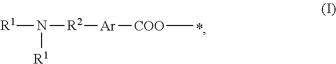

wherein:
Ar is an aromatic heterocycle group comprising at least one nitrogen heteroatom, optionally comprising a sulfur heteroatom;
each R¹ is an organic group of general formula R³—S—R^{3′}, R³—O—R^{3′} or R³ —N—R^{3′};
each R², R³ and R^{3′} are straight chained or branched C₁-C₆-alkyl; and
* is bound to the linker.

In use, it is understood that a compound of general formula (I) includes an R¹ —N—R¹ portion that directly coordinates with the transition metal, in turn drawing electron density from the Ar group towards the complexed R¹—N—R¹ moiety, which weakens the Ar—COO bond and makes the carbonyl group electrophilic. Upon nucleophilic attack on the carbonyl group, the bond between the Ar and COO groups break, resulting in a reaction between the COO group and the nucleophile and formation of a covalent bond, thereby immobilizing the linker (and hence the label portion), whilst the transition metal complex (formed with the metal chelating portion) remains free.

Organic compounds that comprise atoms with lone pairs that are capable of coordinating with a metal atom are known as chelators. The metal chelator portion of the present invention may be, or may comprise, or may consist of, any suitable chelator capable of coordinating to a specific transition metal species. Preferably, the metal chelator portion selectively binds to a single transition metal species. By “selective”, it is envisioned that the metal chelating portion does not bind to any other metal ion at all, or at least does not bind in any significant or substantial amount. The metal chelator portion may be at least about 2 times, or at least about 5 times, or at least about 10 times, or at least about 20 times, or at least about 50 times, or at least about 100 times, or at least about 2, 3, 4, 5, 6, 7, 8, 9, 10, 15, 20, 25, 30, 35, 40, 45, 50, 55, 60, 65, 70, 75, 80, 85, 90, 95, 100 or more times more likely to bind to the selected transition metal species compared to any other metal ion. The binding affinity of the metal chelator portion for the transition metal may be at least about 10⁻¹⁰M, or at least about 10⁻¹¹M, or at least about 10⁻¹²M, or at least about 10⁻¹³M, or at least about 10⁻¹⁴M. In one embodiment, the binding affinity of the metal chelator portion for Cu (I) may be about 10⁻¹³M. In one embodiment of the present invention, the metal chelating portion of the probe may comprise at least one nitrogen and/or at least one sulfur and/or at least one oxygen heteroatom. It may comprise one, two, three or more nitrogen atoms, or it may comprise one, two, three or more sulfur atoms, or it may comprise one, two, three or more oxygen atoms, or it may comprise any combination thereof. As the skilled person would understand, the nitrogen and/or sulfur and/or oxygen heteroatom(s) of the metal chelating portion may form a coordinate bond between the lone pair of the heteroatom (which may be a nitrogen and/or sulfur and/or oxygen atom) and the transition metal. The nitrogen heteroatom may be provided by a primary amine, or a secondary amine or a tertiary amine. The nitrogen heteroatom may be provided by an N-alkylalkylamine, whereby each alkyl group may be independently selected from the group comprising methyl, ethyl, propyl, butyl, pentyl and hexyl (i.e., between C₁ and C₆ alkyl). Each alkyl may be optionally substituted with at least one group consisting of alkyl, alkenyl, halo, hydroxyl, amino, thiol or any other suitable group. The nitrogen heteroatom may be provided by N-ethylethylamine. The sulfur heteroatom, when present, may be provided by an organic sulfide. The organic sulfide may be a dialkylsulfide of general formula alkyl—S—alkyl, whereby each alkyl group may be independently selected from the group comprising methyl, ethyl, propyl, butyl, pentyl and hexyl (i.e., between C₁ and C₆ alkyl). Each alkyl may be optionally substituted with at least one group consisting of alkyl, alkenyl, halo, hydroxyl, amino, thiol or any other suitable group. The organic sulfide may preferably be diethylsulfide. The oxygen heteroatom, when present, may be provided by an ether group of general formula alkyl-O-alkyl, whereby each alkyl group may be independently selected from the group comprising methyl, ethyl, propyl, butyl, pentyl and hexyl (i.e., between C₁ and C₆ alkyl). Each alkyl may be optionally substituted with at least one group consisting of alkyl, alkenyl, halo, hydroxyl, amino, thiol or any other suitable group. The ether may be diethyl ether.

The aromatic heterocycle group may be derived from an aromatic hydrocarbon ring such as benzene, wherein at least one carbon of the heterocyclic ring is substituted by at least one heteroatom. By “heteroatom”, it is meant that an aromatic carbon atom is substituted for a non-carbon atom. Examples of suitable heteroatoms include nitrogen, oxygen and sulfur. The aromatic heterocycle group may be a 4-membered ring, or a 5-membered ring, or a 6-membered ring, or a 7-membered ring, or an 8-membered ring. Accordingly, suitable aromatic heterocycle groups include, for example, pyrrolinyl, pyrrolyl, pyrazolinyl, imidazolinyl, imidazolyl, triazolyl, tetrazolyl, isothiazolyl, thiazolyl, thiadiazolyl, pyridinyl, pyridazinyl, pyrimidinyl, pyrazinyl, triazinyl and thiazinyl, which are respectively radicals derived from the aromatic heterocyclic rings pyrroline, pyrrole, pyrazoline, imidazoline, imidazole, triazole, tetrazole, isothiazole, thiazole, thiadiazole, pyridine, pyridazine, pyrimidine, pyrazine, triazine and thiazine.

The straight chained or branched C₁-C₆-alkyl of R² and/or R³ may be independently selected from the group comprising straight chained alkyl groups methyl, ethyl, propyl, butyl, pentyl and hexyl, or branched chain C₁-C₆-alkyl isomers such as isopropyl, isobutyl, tert-butyl, isopentyl, sec-pentyl, tert-pentyl, isohexyl, and the like.

The organic group, of general formula R³—S—R³ or R³—O—R³, whereby each R³ is a straight chained or branched C₁-C₆-alkyl group as defined herein, may include radicals such as dimethylsulfidyl, methoxymethyl, diethylsulfidyl, ethoxyethyl, dipropylsulfidyl, propoxypropyl, dibutylsulfidyl, butoxybutyl, dipentylsulfidyl, pentoxypentyl, dihexylsulfidyl, hexoxyhexyl, methylethylsulfidyl, methoxyethyl, methylpropylsulfidyl, methoxypropyl, methylbutylsulfidyl, methoxybutyl, methylpentylsulfidyl, methoxypentyl, methylhexylsulfidyl, methoxyhexyl, ethylpropylsulfidyl, ethoxypropyl, ethylbutylsulfidyl, ethoxybutyl, ethylpentylsulfidyl, ethoxypentyl, ethylhexylsulfidyl, ethoxyhexyl, propylbutylsulfidyl, propoxybutyl, propylpentylsulfidyl, propoxypentyl, propylhexylsulfidyl, propoxyhexyl, butylpenthylsulfidyl, butoxypentyl, butylhexylsulfidyl, butoxyhexyl, pentylhexylsulfidyl, pentoxyhexyl, or branched alkyl isomers thereof. Optionally, the organic group may also be substituted with at least one group consisting of alkyl, alkenyl, halo, hydroxyl, amino, thiol or any other suitable substituent group.

In an advantage of the present invention, and as would be appreciated by the skilled person, the oxidation state and ionic radii of a transition metal can affect its coordination geometry. For example, the coordination geometry of Cu(I) is tetrahedral and Cu(II) is distorted square pyramidal, and the ionic radii of Cu(I) is 77 pm, compared to 73 pm for Cu(II). As the skilled person would appreciate, chelators can be designed so that the heteroatoms of the chelator are arranged to line up with, or substantially align with, the coordination geometry and ionic radii of a particular transition metal ion, whereas the same chelator is unable to form coordination bonds with other transition metal ions, due to a misalignment of the coordination geometry or other steric hindrances. Accordingly, the metal chelating portion of the probe may be adapted to specifically complex with a specific transition metal in a select oxidation state. By way of an example, the metal chelating portion of the probe may be adapted to preferentially bind to Cu(I) over Cu(II), or any other metal atom. It is particularly preferred that common biological ions such as Mg²⁺, Ca²⁺, Na⁺and K⁺ do not bind to the metal chelating probe of the present invention.

In one embodiment, the metal chelating portion is according to formula (II):

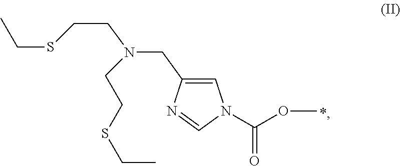 [SMILES: CCSCCN(CCSCC)Cc1cn(C(=O)OC)cn1]

wherein * indicates the binding site with the linker. It is understood that in the metal chelating portion of formula (II), a Cu(I) ion forms coordination bonds with the two sulfur heteroatoms and the two nitrogen atoms that are not involved in bonding to the linker.

### C. Label Portion

The label portion of the present invention is adapted to be detected or is detectable. It is preferably inherently adapted to be detected and does not required ‘activation’ via binding with the transition metal in order to be detectable. The label is physically distanced from the metal chelating portion of the probe by the linker. Accordingly, the complexing of the transition metal to the metal chelating portion is not required in order for the label portion to be detectable.

The label portion of the probe of the present invention may be detected, or may be detectable, by any suitable technique. For instance, the label portion may be adapted to be detected by, or detectable by, at least one of fluorescence, PET imaging, medical resonance imaging (MRI) computed tomography (CT) imaging or synchrotron X-ray imaging techniques. Accordingly, the label portion may comprise at least one of a fluorophore, a luminescent marker, a radiolabel (i.e., a positron emitter), a gadolinium(III) complex, a metal nanoparticle, a barium complex, or an iodine-containing compound, or any combination thereof. The label portion may be detected, or detectable, by more than one technique. It may be detectable by two or three or four techniques or more. For example, the label portion may comprise a fluorophore and a radiolabel, allowing the label portion to be detected by both fluorescence and PET imaging, which allows for use of the probe in both a living subject and in a post mortem tissue or tissue sample.

The fluorophore, when present, may be any organic compound that is able to absorb light at a particular wavelength and then emit light at a higher wavelength. The fluorophore may comprise a single aromatic ring or two or more fused aromatic rings (i.e., a conjugated system). The fluorophore may be based on, or be a derivative of, naphthalene, coumarin, xanthine, anthracene, pyrene, tetrapyrrole, oxadiazole, oxazine or acridine. By “derivative”, it is meant that any fluorophore that comprises a ring system described herein may be used, so long as the derivative is a fluorophore (i.e., capable of fluorescing). In one embodiment, the fluorophore may be a naphthalimide fluorophore or a derivative thereof. It may be naphthalimide. It may be 4-amino-1,8-naphthalimide or a substituted derivative thereof.

The radiolabel, when present, may be a positron emitter (i.e., it may be radioactive and undergoes beta plus (β⁺) decay) or an electron emitter (i.e. it may be radioactive and undergoes beta minus (β⁻) decay). The radiolabel may be any one of ¹¹C, ¹³N, ¹⁵O, ¹⁸F, ⁷⁸Br, ¹²⁴I, ¹³¹I or any other suitable radionuclide capable of positron emission or electron emission. In one embodiment, it may be ¹⁸F. When the label portion comprises or consists of a fluorophore and a radiolabel, the radiolabel may be a substituent group covalently bound to the fluorophore or it may be a heteroatom within the fluorophore.

In one embodiment, the fluorophore may be the naphthalimide derivative of formula (III):

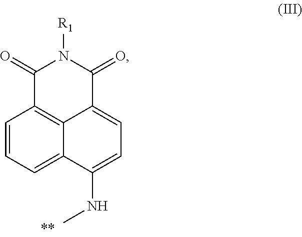

wherein
R⁴ is optionally substituted C₁-C₆ alkyl and ** is bound to the linker, wherein alkyl is as defined herein.

The C₁-C₆ alkyl may be substituted with one or more substituents selected from amino, carboxyl, C₃-C₆ aryl and halo, wherein the aryl may be further substituted with one or more substituents selected from amino, carboxyl, C₁-C₆alkyl, C₃-C₆ aryl and halo. Each alkyl may contain 0, 1, 2 or 3 heteroatoms selected from a nitrogen atom, an oxygen atom and a sulfur atom. R⁴ may also be substituted so as to comprise a radiolabel, such as a radionuclide. In one embodiment, the label portion may comprise, or consist of, the naphthalimide derivative of formula (III) above, wherein R⁴ is ethyl, wherein the ethyl is further substituted with the radiolabel ¹⁸F. Accordingly, the label portion may be formula (IIIa):

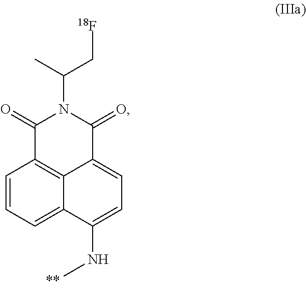 [SMILES: CNc1ccc2c3c(cccc13)C(=O)N(C(C)C[18F])C2=O]

wherein ** is bound to the linker.

In an alternative embodiment, the fluorophore may be the naphthalimide derivative of formula (IV):

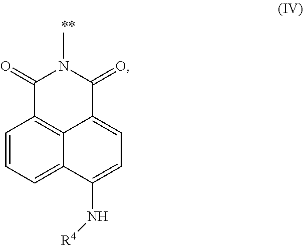

wherein R⁴ is optionally substituted C₁-C₆ alkyl and ** is bound to the linker, wherein alkyl is as defined herein.

The C₁-C₆ alkyl may be substituted with one or more substituents selected from amino, carboxyl, C₃-C₆ aryl and halo, wherein the aryl may be further substituted with one or more substituents selected from amino, carboxyl, C₁-C₆ alkyl, C₃-C₆ aryl and halo. Each alkyl may contain 0, 1, 2 or 3 heteroatoms selected from a nitrogen atom, an oxygen atom and a sulfur atom. R⁴ may also be substituted so as to comprise a radiolabel, such as a radionuclide.

Without being bound to theory, the inventors do not expect that the orientation of the naphthalimide fluorophore (that is, whether the linker is bound to the imide nitrogen or the amine nitrogen) affects the overall mechanism of action of the probe (that is, immobilisation of the label portion) and that the two orientations (based on either general formulae (III) and (IV)) are interchangeable.

### D. Linker

The linker of the probe described herein is located between the metal chelating portion and the label portion. It is covalently bound to both the metal chelating portion and the label portion as described above. Preferably, the linker portion separates the metal chelating portion and the label portion by being positioned between these two portions. In other words, it is preferred that the metal chelating portion is bound at one end of the linker and the label portion is bound at the other end of the linker (i.e., [metal chelating portion]-(linker)-[label portion]), although alternative arrangements may be used. The linker may be C₁-C₆ alkyl, whereby the alkyl may be straight chained or branched C₁-C₆ alkyl and may further comprise 0, 1, 2 or 3 heteroatoms selected from a nitrogen atom, an oxygen atom and a sulfur atom.

The linker of the present invention may be any suitable carbon-containing group. It may be a straight chained or branched C₁-C₆ alkyl, or a C₂-C₆ alkenyl, or a C₂-C₆ alkynyl group, or an aryl group as defined above. It may be optionally substituted with 1, 2 or 3 heteroatoms selected from a nitrogen atom, a sulfur atom and an oxygen atom. It may be optionally substituted with at least one group consisting of alkyl, alkenyl, halo, hydroxyl, amino, thiol or any other suitable group. It may be of general formula —R⁵—, or —R⁵—N−R⁵, or −R⁵—O—R⁵, or −R⁵—S—R⁵, wherein each R⁵ may be a straight chained or branched C₁-C₆ alkyl, or a C₂-C₆ alkenyl, or a C₂-C₆ alkynyl group, or an aryl group. In one embodiment, R⁵ may be selected from methyl, ethyl, propyl or butyl. In one embodiment, the linker may be —CH₂—CH₂—O—CH₂—CH₂—. In another embodiment, the linker may be —CH₂—CH₂—.

In view of the above, the probe of the present invention may be selected from the compounds:

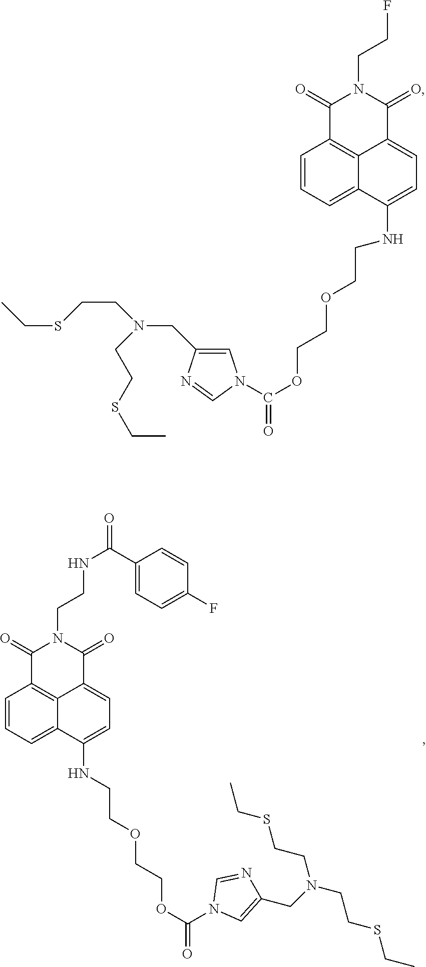

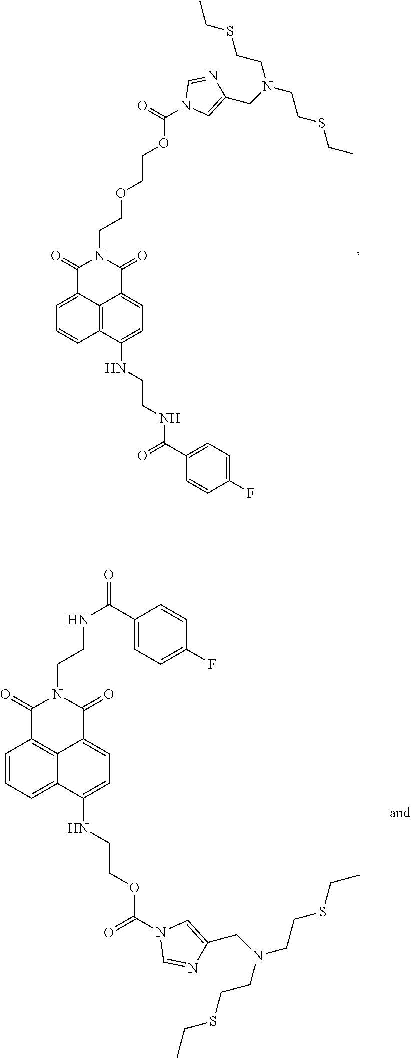

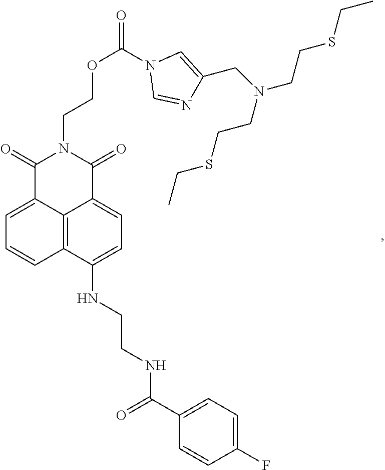

wherein F is ¹⁸F or ¹⁹F.

### III. In Vivo Use

Once produced, the probe of the present invention as described herein may be used to identify, and/or quantify, selected transition metals in vivo (i.e., in a living subject, without taking a sample for analysis outside of the subject), which provides an advantage to the present invention over prior art chelators or transition metal probes. In particular, as described herein, the metal chelating portion of the probe may be adapted to selectively bind to only ions of a specific transition metal, including specifically to a certain oxidation state of that transition metal element. Once a particular transition metal ion is ‘recognised’, the covalent bond between the metal chelating portion and the linker is weakened, allowing a reaction between the reactive carboxyl group of the linker and a nearby nucleophile such as a protein or other intracellular or extracellular structure, effectively immobilising the label portion (which is inherently adapted to be detected, or is detectable, by known imaging or analytical techniques).

Chelators with specific selectivity are known in the art. There are also compounds in the art known to form complexes with specific transition metal species, the result of which is that the transition metal complex is then fluorescent, whereas the chelator on its own is ‘off’ (i.e., does not fluoresce). Put differently, binding of the transition metal to the ‘off’ or nonfluorescent compound turns the complex into a fluorophore, essentially ‘switching on’ its fluorescence and hence detectability when bound. However, such fluorescent complexes are still free to diffuse through tissues or fluids (meaning that a static picture of transition metal distribution and/or concentration cannot be obtained unless the tissue or cell sample is fixed) and the fluorescent complex cannot be detected, or is not detectable, in a living subject due to the reliance of this technique on visible light.

The probe of the present invention has been developed to ameliorate at least one of these deficiencies. In particular, the probe as described herein has been designed to measure transition metal location(s) and/or concentration(s) in vivo in a living subject (as opposed to an ex vivo tissue sample, or a sample obtained post mortem). A method for measuring a transition metal in vivo may include, or comprise, administering to a subject an effective amount of the probe as described herein and then detecting the label portion. Measuring the amount and/or location of a transition metal in a subject may involve the following aspects:
Immobilisation of the label portion at the site of a transition metal ion recognition, allowing for an accurate measurement of transition metal distribution and concentration that is not
affected by circulation or other metabolic or biological processes, such as elimination;
The label portion is adapted to be detected by, or is detectable by, known medical imaging and/or analytical techniques, including both in a living subject and in an ex vivo tissue sample or post mortem tissue sample; and
The properties of the probe can be adjusted to target different compartments of the subject, for instance the log P, size and physiological charge can be adjusted to allow the probe to cross the blood brain barrier for measurement of the transition metals in the central nervous system.

Accordingly, the probe of the present invention may be used for measuring a transition metal in vivo by detecting the immobilized label portion in a subject after administration of the probe to the subject. The measuring may be indirect (i.e., the transition metal per se is not measured, but the immobilised label portion is measured at the location that the transition metal was recognised). By “measuring”, it is meant that the immobilised label portion is detected, and at least one aspect of the immobilised label portion is measured and recorded. For instance, the location of at least one immobilised label portion relative to the biological structures of the subject or the tissue may be measured and recorded, or the location of two or more immobilised label portions may be measured and recorded, which may be extrapolated to infer, or identify, biological structures or regions of relatively high, or relatively low, concentration based on the relative intensity of the measured immobilised label portions. In other words, local or general concentrations of specific transition metals may be determined by using the probe of the present invention and measuring, or detecting, the immobilised label portions.

Accordingly, the probe of the present invention may also be used to identify regions of metal dyshomeostasis in a subject. As the skilled person would appreciate, by “dyshomeostasis” it is meant that there is an imbalance or other breakdown in a homeostatic system. In regard to the present invention, “metal dyshomeostasis” refers to the imbalance or breakdown of a homeostasis system for maintaining the amount of at least one transition metal in a subject, either systemically or locally in a particular biological compartment, organ, tissue or structure. Regions of metal dyshomeostasis in a subject may be determined by use of a method whereby:
a subject is administered an effective amount of the probe described herein;
the location of at least one immobilised label portion, or more preferably the location of two or more immobilised label portions, of the probe are measured in a subject or a tissue sample to identify biological structures or regions of high, and/or low, concentration of the immobilised label portion (which is understood to be relative to transition metal concentration); and
the location of the immobilised label portion(s) in the subject are compared with the location and/or concentrations of the same transition metal in the same biological structures or regions of a control subject that has normal metal homeostasis, whereby any region(s) or structure(s) or tissue(s) with some variation, or substantial variation, or significant variation, from a control subject may be considered to be indicative of regions of metal dyshomeostasis.

The subject may be a mammal. The subject may be a human or it may be an animal (i.e., the probe of the present invention may be suitable for use in medicine or in veterinary medicine). The animal may be a companion animal (such as a cat or a dog), or it may be a livestock animal (such as a cow, a sheep, a goat, a pig, or poultry) or it may be any other animal. In one embodiment, the subject may be a dog.

The region, or biological structure, or tissue, identified as being a region of metal dyshomeostasis in a subject, may be any suitable biological location. For example, as used herein: ‘region’ may refer to a general area or system of the subject and may include, for instance, the central nervous system (or a part thereof), the abdomen (or a part thereof), the gastrointestinal tract (or a part thereof) or the lymphatic system (or a part thereof); ‘biological structure’ may refer to any suitable macrostructure that comprises multiple tissue types (such as, for instance, an organ) or any suitable microstructure (such as, for instance, a cell, or an intracellular organelle or protein) that are associated with normal metal homeostasis; and ‘tissue’ may refer to any suitable tissue of the subject that may contribute to metal homeostasis, or requires the transition metal as an essential metal, such as, for example, the brain, neurons, liver, spleen, intestines, pancreas, kidneys and the like. In other words, it is envisioned that the probe of the present invention can be adapted to measure a transition metal location and/or concentration and/or speciation and/or its oxidation state in any particular tissue, organ or system of the subject or region thereof, particularly in a living subject. In one embodiment, the region of the subject investigated for metal dyshomeostasis is the central nervous system. The region may comprise, or the focus of the measurement of the immobilised label portion, may be the brain of the subject. When a region of the central nervous system, such as the brain or the spinal cord, is identified as being a region of metal dyshomeostasis in the subject (i.e., whereby a specific portion of the central nervous system has a relatively high, or a relatively low, area of transition metal concentration compared to a control subject without a metal dyshomeostasis), this region may be indicative of a neurological condition. The neurological condition may be any suitable condition associated with an imbalance of the measured transition metal in the particular location that the imbalance is identified in. For example, if the transition metal is copper, the region or regions of copper dyshomeostasis (which may be indicated as being a region of relatively high, and/or relatively low, copper concentration) in the central nervous system, such as the brain or spinal cord, may be indicative of any one of Parkinson's disease, Alzheimer's disease, amyotrophic lateral sclerosis (ALS), Wilson's disease, multiple sclerosis (MS) and Menkes disease, or it may be a cancer, such as neuroblastoma. It is expected that the skilled person would associate certain region(s) of metal dyshomeostasis in the central nervous system to certain neurological conditions; for example, a region of relatively high copper (i.e., a copper excess) in the entorhinal cortex or hippocampus of the brain may be indicative of Alzheimer's disease, generalised regions of low copper (i.e., a copper deficiency) across the brain may be indicative of Menkes disease, a region of relatively high iron may be indicative of stroke, or a relative deficiency of soluble copper in the ventral spinal cord compared to the dorsal spinal cord may be indicative of ALS. In another embodiment, the region of the subject investigated for metal dyshomeostasis is the endocrine system. The region may comprise, or the focus of the measurement of the immobilised label portion, may be the pancreas of the subject. For instance, a deficiency of zinc and/or chromium in the pancreas, which are known to be required in the production of insulin, may be indicative of diabetes mellitus. In yet another embodiment, the region of the subject investigated for metal dyshomeostasis is the digestive system. The region may comprise, or the focus of the measurement of the immobilised label portion, may be the liver of the subject. For instance, excessive amounts of transition metals such as copper, arsenic, zinc and chromium (particularly Cr(IV)) may cause liver damage and lead to liver disease, or accumulation of copper in the liver of the subject may be indicative of Wilson's disease in a human, or copper storage hepatopathy in a dog, for instance.

As the skilled person would appreciate, it follows that another use for the probe of the present invention is in a method of diagnosing a condition associated with metal dyshomeostasis. For example, as the neurons and tissues of the central nervous system relies on particular transition metals, the probe of the present invention may be used in a method of diagnosing a neurological condition in a subject. Similarly, organs such as the liver and pancreas may also accumulate transition metals that may be diagnostically relevant in identifying conditions such as cirrhosis and diabetes mellitus.

A diagnostic method may comprise the steps of administering to both a subject (i.e., a subject suspected of suffering from metal dyshomeostasis, or a condition associated with metal dyshomeostasis) and a control subject (i.e., a subject considered to have normal metal homeostasis) an effective amount of the probe as described herein. The location of the immobilized label portions, and the concentration of the label portions in corresponding region(s) in both the subject and the control subject, may be determined by detecting the immobilized label portion via the technique(s) that the label portion has been adapted to be detected by, or is detectable by. The detected label portion(s) may be used to form a subject map of label intensity, and a control map of label intensity. The control map may be formed at about the same time as the subject map, or it may be formed earlier than the subject map, or it may be formed later than the subject map; in other words, the subject and the control subject may be administered the probe and the label portion measured simultaneously, or nearly simultaneously, or roughly simultaneously (i.e., at about the same time, such as on the same day) or a control map may be initially produced as a standard by which all other subject maps are compared. The control map may comprise measurements taken from one subject, or they may comprise measurements taken from more than one control subject and averaged.

The next step may comprise comparing the subject map and the control subject map and identifying regions of relatively high concentration in the subject map, and/or relatively low concentration in the subject map. Where the difference, or variance, between the subject map and the control subject is more than about 5% intensity, or more than about 10% intensity, or more than about 20% intensity, or more than about 50% intensity, or more than about 75% intensity, or more than about 90% intensity, more than about 95% intensity, more than about 100% intensity, or about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% or more, the difference or variance may be indicative of a metal dyshomeostasis condition in the subject. The biological location of the difference or variance of transition metal location may provide a physician or other medical professional with information relative to the diagnosis of the condition. For instance, if the immobilized label portion is located in the central nervous system, or the brain or a region or area thereof, in the subject (and the control subject), areas or regions of relatively high (or low) transition metal concentration, such as copper, may be indicative of, or provide for the diagnosis of, a neurological condition. The neurological condition may be stroke, Parkinson's disease, Alzheimer's disease, amyotrophic lateral sclerosis (ALS), Wilson's disease, multiple sclerosis (MS), Menkes disease, cancer (such as neuroblastoma) or another suitable condition or disease. Likewise, if the immobilized label portion is located in the pancreas, and relatively high levels of copper are identified in a subject when compared to a control subject, this may be indicative of, or provide for the diagnosis of, diabetes mellitus. Further, the skilled person would be aware that cancer can also affect the local concentration of transition metal and so regions of relatively high or low concentration may be indicative of the location of a solid cancer. Furthermore, some tumours are known to accumulate copper before rapidly growing, hence the probe of the present invention may be used to monitor tumours and identify those with the potential to begin rapid growth.

The skilled person would also appreciate that the probe of the present invention may be used to identify a subject that has excessively high levels of a transition metal that is not an essential element (i.e., with no beneficial biological function). By way of example, the probe may be adapted to bind to Cr(VI), which is a known toxicant and carcinogen and it may be selective for Cr(VI) over Cr(III). Similarly, the probe may be adapted to bind to Hg(I) or Hg(II) to identify regions of bioaccumulation in subjects exposed to mercury vapour.

The in vivo methods or uses described herein may further require a period of time between administering the probe to the subject (or the control subject) and measuring the location of the immobilized label. As the skilled person may appreciate, there may be a period of time required after administering the probe to allow for the probe (or a significant amount of the administered probe) to be absorbed, and/or transported to the region or tissue that the probe is adapted to reach or accumulate in, and/or recognise a transition metal that the metal chelating portion has been adapted to complex with, and then immobilize the label portion whilst eliminating any excess (i.e., unreacted) probe (as, in most embodiments, the detector cannot distinguish between an immobilized label portion and a mobile label portion since the label portion does not need to be switched ‘on’). The portion of time may be between about 1 minute and about 2 hours, or between about 3 minutes and about 1 hour, or between about 5 minutes and about 30 minutes, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119 or 120 minutes, or any range therein.

The in vivo methods or uses described herein require the administration of an effective amount of the probe to the subject. The administration may be by any suitable method, which may depend on the tissues or organs of the subject that the probe is adapted to reach or accumulate in. For example, the administration to the subject may be by enteral administration (such as oral administration, nasal administration, sublingual administration or buccal administration), or parenteral administration, (such as by injection or infusion, for example by intravenous injection or infusion, or by subcutaneous injection or infusion, or by intramuscular injection or infusion), or by intranasal administration or by inhalation. The probe may be administered in any dosage form suitable for the administration route. For instance, the probe may be: in a sterile solution or dispersion formulation for injection, infusion, nebulization or aerosolization; or in a potable liquid (such as a syrup or elixir) or a solid dosage form (such as a tablet, lozenge, wafer, chew or capsule) for oral administration; or a powder for inhalation. In one embodiment, the probe of the present invention is administered as a sterile solution by intravenous infusion.

### IV. Ex vivo Use

The probe of the present invention may also be used to detect, or locate, or measure, transition metals in ex vivo samples (i.e., samples removed from the subject and analysed or measured outside the body). The ex vivo sample may be taken from a subject before, during, or after the label portion has been detected in the subject by a medical imaging technique. The ex vivo sample may be a post mortem sample. The ex vivo sample may be detected by the same technique that the in vivo label portion was detected, or it may be a different technique. As an example, the label portion of the probe may be adapted to be detectable by a lab-based analytical technique (such as, for example, fluorescence; that is, the label portion comprises a fluorophore) as well as by a medical imaging technique (such as, for example, PET imaging; that is, the label portion may also comprise a radiolabel, such as ¹⁸F), which would allow for analysis of the same immobilised label portions, but by two different techniques.

The probe may also be used in a method for labelling, or detecting, or measuring, a transition metal in a biological tissue sample. The sample may be taken from a subject without a prior step of administering the probe to the subject. The sample may be contacted with the probe outside the subject, whereupon the metal chelating portion of the probe recognises and binds to the transition metal in the biological sample, resulting in the cleavage of the metal chelating portion from the linker and immobilisation of the label portion in the sample. In other words, it is understood that the immobilisation of the label portion in a biological sample does not require the probe to contact the transition metal in a living subject, but can occur in any suitable biological sample that comprises at least (1) a transition metal for which the metal chelating portion of the probe is adapted to bind to, and (2) a nucleophile capable of reacting with the carboxyl group of the linker after the transition metal complexes, or coordinates, with the metal chelating portion. The nucleophile in the ex vivo sample may be the same as described above for the in vivo sample. It may be an amino acid or an amino acid sidechain. The amino acid may be located in a protein or peptide.

### V. Examples

The present invention will be further described by way of the following illustrative examples provided to assist the skilled person in understanding the present invention, and which are not intended to the limit the present invention.

### Example 1: General Synthesis

A series of reactions were conducted, as set out in Scheme 1 below, to make the compound provided above as formula (IV), also referred to herein as F—NpCul.

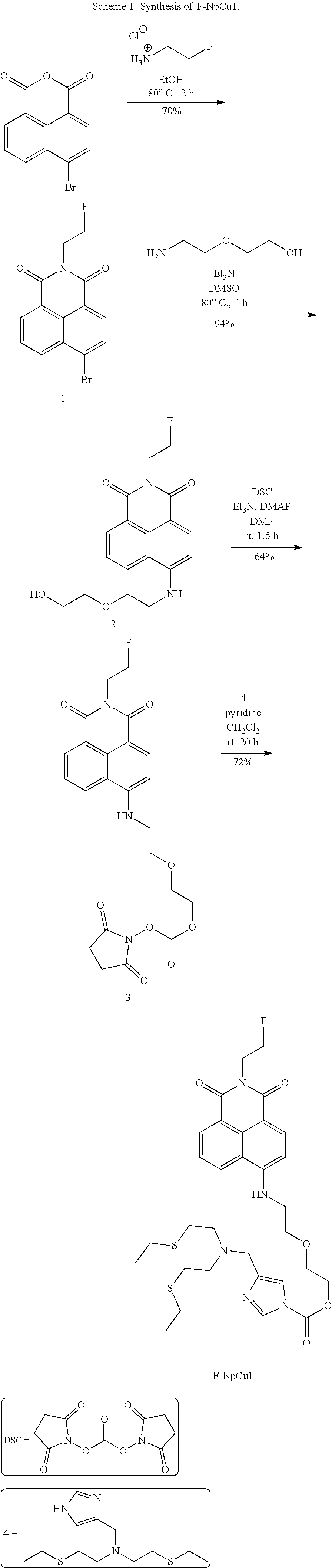

All reactions were performed under a nitrogen atmosphere. All reagents were used as received unless otherwise specified. Dichloromethane (CH₂Cl₂) and N,N-dimethylformamide (DMF) were obtained from a Pure-Solv 400 Solvent Purification System. All other solvents were laboratory grade and used without further purification. Reactions were monitored by silica gel thin-layer chromatography plates (Merck, TLC Silica gel 60 F254). All column chromatography was performed on silica gel 60 (Merck, 0.040-0.063 nm) in 12 g or 25 g iLok cartridges (Santai Technologies) using a Biotage Isolera One. Copper sensing group (compound 4 of Scheme 1) was synthesised according to previously reported procedures.

Each synthesized compound of Scheme 1 was characterised. All NMR spectra were obtained at 300 K on Bruker AVANCE III 400 or Bruker AVANCE III 500 spectrometers equipped with a 5 mm BBFO probe with z-gradients. Deuterated solvents (CDCl₃, DMSO-d₆) were obtained from Cambridge Isotope Laboratories. All chemical shifts are reported in ppm and all coupling constants are reported in Hz. ¹H NMR spectra are calibrated to trace isotopic impurities of the solvent used (δ=7.26 ppm for CDCl₃, δ=2.50 ppm for DMSO-d₆). ¹H NMR data are reported as: chemical shift, multiplicity, coupling constant(s) (J) and relative integral. The multiplicities are reported as one or more of the following: s=singlet, d=doublet, t=triplet, q=quartet, quint=quintet, m=multiplet, br=broad. ¹³C {¹H} NMR spectra are calibrated to trace isotopic impurities of the solvent used (δ=77.16 ppm for CDCl₃, δ=39.52 ppm for DMSO-d₆). ¹³C {¹H} NMR data is reported as chemical shift.

Low resolution ESI and APCI mass spectrometry was performed on a Bruker AmaZon SL ion trap mass spectrometer. For low resolution ESI, samples were injected via flow injection at 0.3 mL/min in methanol or acetonitrile into an Apollo II source with nitrogen drying gas at 180° C. For low resolution APCI, samples were placed in a melting point tube and inserted into the Bruker Apollo II APCI source with an atmospheric solid analysis probe attachment added with vaporisation temperature 400° C. and corona current 4 μA. High resolution ESI and APCI mass spectrometry was performed on a Bruker solarix 2XR Fourier Transform Ion Cyclotron Resonance Mass Spectrometer. For high resolution ESI, samples were injected using the supplied syringe pump at 180 μL/h with nebuliser flow 1 L/min and drying gas 4L/min at 180° C. For high resolution APCI, samples were placed in a melting point tube and inserted into the Bruker Apollo II APCI source with an atmospheric solid analysis probe attachment added with vaporisation temperature 400° C. and corona current 4 μA.

### Synthesis and Characterisation of Compound 1

4-Bromonaphthalic anhydride (1.00 g, 3.61 mmol) and 2-fluoroethylamine hydrochloride (0.538 g, 5.41 mmol) were suspended in absolute EtOH (50 mL). Et₃N (1.0 mL, 7.2 mmol) was added and the suspension heated to 80° C. for 20 min, after which the reaction mixture became a solution. After 2 h, a new precipitate had formed. The reaction mixture was cooled, and the precipitate isolated by filtration and washed with cold EtOH then dried. This afforded 1 (0.812 g, 70%) as a beige powder.

¹H NMR (400 MHZ, CDCl₃): 8.68 (dd, J=7.3, 1.0 Hz, 1H), 8.60 (dd, J=8.6, 1.1 Hz, 1H), 8.44 (d, J=7.9 Hz, 1H), 8.06 (d, J=7.9 Hz, 1H), 7.86 (dd, J=8.6, 7.3 Hz, 1H), 4.75 (dt, J=47.0, 5.2 Hz, 2H), 4.57 (dt, J=23.3, 5.2 Hz, 2H).

¹³C NMR (101 MHZ, CDCl₃): 163.9, 163.8, 133.7, 132.5, 131.7, 131.3, 130.9, 130.8, 129.3, 128.3, 123.0, 122.1, 81.0 (d, J=167 Hz), 40.4 (d, J=22 Hz).

HRMS (ESI) m/z: [M+H] ⁺ Calcd for C₁₄H₁₀BrFNO₂⁺ 321.9874 and 323.9854; Found 321.9873 and 323.9852.

### Synthesis and Characterisation of Compound 2

Compound 1 (0.40 g, 1.2 mmol) and triethylamine (0.52 mL, 3.7 mmol) were suspended in DMSO (5 mL). The reaction mixture was heated to 60° C. for 15 min. After the solids dissolved, 2-(2-aminoethoxy) ethanol (0.38 mL, 3.7 mmol) was added dropwise and reaction mixture was heated at 80°° C. for 16 h. The reaction mixture was diluted with water (200 mL) and compound was extracted with CH₂Cl₂ (3× 150 mL). The combined organics were washed with brine (400 mL), dried over Na₂SO₄ and dried to give 2 as a yellow solid (0.41 g, 94%).

¹H NMR (400 MHZ, DMSO-d₆): 8.68 (d, J=8.3 Hz, 1H), 8.43 (d, J=7.3 Hz, 1H), 8.25 (d, J=8.3 Hz, 1H), 7.76 (t, J=5.4 Hz, 1H), 7.67 (dd, J=8.3, 7.7 Hz, 1H), 6.82 (d, J=8.7 Hz, 1H), 4.66 (t, J=5.0 Hz, 1H) 4.65 (dt, J=47.3, 5.3 Hz, 2H), 4.36 (dt, J=22.9, 5.3 Hz, 2H), 3.73 (t, J=5.5 Hz, 2H), 3.57 (q, J=5.4 Hz, 2H), 3.54-3.48 (m, 4H).

¹³C NMR (101 MHz, DMSO-d₆): 163.9, 162.9, 150.8, 134.4, 130.9, 129.5, 128.7, 124.4, 121.7, 120.1, 107.5, 104.1, 80.0 (d, J=167 Hz), 72.3, 68.1, 60.3, 42.9.1×C under solvent peak confirmed by HSQC.

HRMS (ESI) m/z: [M+Na] ⁺ Calcd for C₁₈H₁₉FN₂NaO₄⁺ 369.1221; Found 369.1222.

### Synthesis and Characterisation of Compound 3

Compound 2 (100 mg, 0.289 mmol), N,N′-disuccinimidyl carbonate (DSC) (185 mg, 0.722 mmol) and dimethylaminopyridine (DMAP) (3 mg) were dissolved in N,N-dimethylformamide (2 mL) and stirred at room temperature for 1.5 hours. The reaction mixture was then diluted with EtOAc (30 mL) and washed with 5% w/v LiCl solution (5×30 mL). The organic extract was dried over Na₂SO₄ and concentrated in vacuo. Purification by column chromatography (silica gel, 40-60% EtOAc in hexanes) afforded 3 (90 mg, 64%) as a yellow solid.

¹H NMR (400 MHZ, CDCl₃): 8.58 (d, J=7.3 Hz, 1H), 8.46 (d, J=8.3 Hz, 1H), 8.22 (d, J=8.6 Hz, 1H), 7.61 (t, J=8.3 Hz, 1H), 6.72 (d, J=8.6 Hz, 1H), 5.89 (t, J=4.8 Hz, 1H), 4.74 (dt, J=47.1, 5.2 Hz, 2H), 4.59-4.51 (m, 4H), 3.89 (t, J=5.2 Hz, 2H), 3.86-3.84 (m, 2H), 3.62 (q, J=5.2 Hz, 2H), 2.79 (s, 4H).

¹³C NMR (101 MHZ, CDCl₃): 168.1, 165.0, 164.3, 152.0, 149.8, 134.8, 131.5, 130.1, 127.0, 124.9, 122.9, 120.8, 110.4, 104.6, 81.4 (d, J=169 Hz), 70.0, 69.0, 68.6, 43.2, 40.0 (d, J=23 Hz), 25.6.

HRMS (ESI) m/z: [M+Na] +Calcd for C₂₃H₂₂FN₃NaO₈⁺ 510.1283; Found 510.1283.

### Synthesis and Characterisation of F—NpCul

A solution of compound 4 (33 mg, 120 μmol) in CH₂Cl₂ (1 mL) was added dropwise to a stirring solution of compound 3 (60 mg, 0.12 mmol) in CH₂Cl₂ (5 mL). Anhydrous pyridine (35 μL, 0.43 mmol) was added and the reaction mixture stirred at room temperature for 20 hours. The reaction mixture was diluted with 5% w/v citric acid solution (20 mL) and the product extracted with CH₂Cl₂ (2×15 mL). The combined organics were washed with brine (30 mL), dried over Na₂SO₄ and concentrated in vacuo. Purification by column chromatography (silica gel, 60-100% EtOAc in hexanes) afforded F—NpCul (57 mg, 72%) as a yellow oil.

¹H NMR (400 MHZ, CDCl₃): 8.57 (dd, J=7.3, 0.8 Hz, 1H), 8.44 (d, J=7.4 Hz, 1H), 8.11 (d, J=8.1 Hz, 1H), 8.07 (d, J=1.3 Hz, 1H), 7.61 (dd, J=8.4, 7.4 Hz, 1H), 7.34 (d, J=0.9 Hz, 1H), 6.70 (d, J=8.5 Hz, 1H), 5.72 (t, J=4.9 Hz, 1H), 4.73 (dt, J=47.5, 5.3 Hz, 2H), 4.64-4.61 (m, 2H), 4.54 (dt, J=22.9, 5.3 Hz, 2H), 3.92-3.89 (m, 4H), 3.64 (s, 1H), 3.60 (q, J=5.0 Hz, 2H), 2.76-2.72 (m, 4H), 2.65-2.61 (m, 4H), 2.51 (q, J=7.4 Hz, 4H), 1.22 (t, J=7.4 Hz, 3H).

¹³C NMR (101 MHZ, CDCl₃): 164.8, 164.2, 149.5, 148.9, 141.9, 136.9, 134.7, 131.6, 130.0, 136.33, 125.0, 123.0, 120.6, 115.1, 110.6, 104.6, 81.3 (d, J=171 Hz), 68.9, 66.7, 54.0, 51.2, 43.2, 39.9 (d, J=23 Hz), 29.6, 26.4, 15.0.

HRMS (ESI) m/z: [M+H] ⁺ Calcd for C₃₁H₄₁FN₅O₅S₂⁺ 646.2528; Found 646.2527.

### Photophysical Studies

For all studies, a 10 mM stock solution of F—NpCul in DMSO was diluted to the appropriate concentration in phosphate buffered saline (PBS, 10 mM, pH=7.4). The DMSO concentration in all experiments was <0.5% v/v.

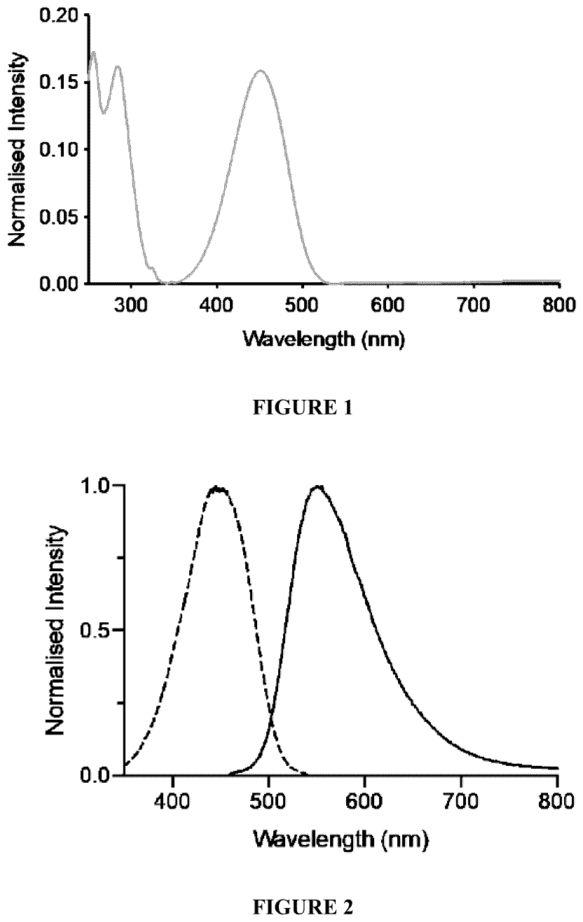

The absorption spectrum (FIG. 1) was obtained on Varian Cary 400 UV-Vis spectrophotometer using 10 mm pathlength reduced-volume quartz cuvettes. The fluorescence spectrum (FIG. 2) was obtained on a Horiba Duetta fluorescence and absorbance spectrometer using 10 mm pathlength reduced-volume quartz cuvettes.

### Example 2: In Vitro and Iv Vivo Testing

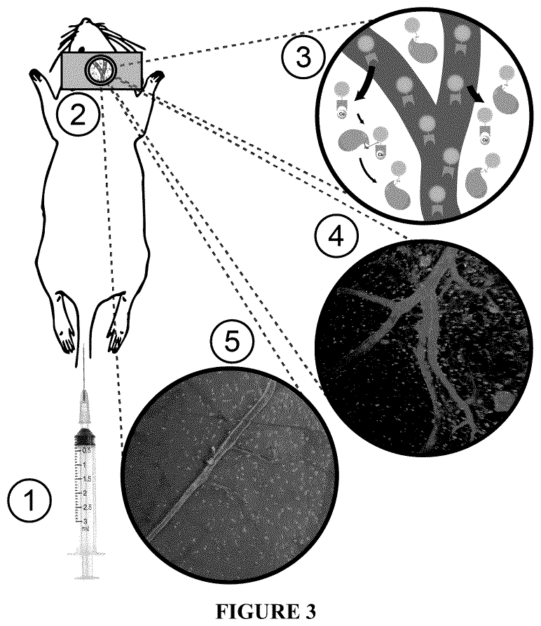

Preliminary results have been obtained to show that F—NpCul is bioavailable and can be taken up by cells to report intracellular Cu(I) levels, and that the intensity of the signal corresponds to copper-loading or copper-deficiency. In this regard, FIG. 3 shows a method of quantifying cortical Cu levels in the intact mouse brain with the Deep In Vivo Explorer (DIVE) multiphoton microscope. Following intravenous injection of a fluorescent dextran tracer (1), the fluorescent tracer was detected in the blood vessels (red; 4) of the parietal cortex (2). Subsequent retro-orbital injection of F—NpCul enabled real time 3D imaging of Cu levels via Cu-recognition and accumulation of the Cu-activated F—NpCul-protein complex (shown schematically in (3)) in the brain tissue (green; 5) demonstrating that F—NpCul is readily permeable through the blood brain barrier.

### Cell Culture

SH-SY5Y cells were used, which are a human neuron-like cell line. SH-SY5Y cells were maintained at 37° C. in 5% CO₂. Cells were cultured in Dulbecco's Modified Eagle's Medium (DMEM, Thermo Fisher Scientific) supplemented with 10% foetal calf serum (FCS, Thermo Fisher Scientific) and 2.5 mM L-glutamine (Sigma-Aldrich). Fluorobrite DMEM (FDMEM, Thermo Fisher Scientific) was supplemented with 10% FCS and 2.5 mM L-glutamine, unless otherwise stated. Cells for imaging experiments had a passage number lower than 20.

### Confocal Microscopy

For all imaging experiments, 1.5×10⁵ cells were seeded into 35 mm glass bottom dishes (MatTek) and allowed to adhere overnight. 10 mM stock solutions of F—NpCul CuATSM were freshly prepared in DMSO. A 10 mM stock solution of BCS was freshly prepared in MilliQ water. For experiments cells were dosed with nothing (vehicle), 1 μL of 10 mM CuATSM stock in 1 mL DMEM or 10 μL of 10 mM BCS solution in 1 mL DMEM. Cells were incubated for 1 hour, and 1 μL of F—NpCul stock solution was added to each dish and incubated for 20 minutes. Cells were washed with PBS (3×0.5 mL) and suspended in FDMEM for imaging. All cells were imaged immediately after washing.

Images were obtained at 37° C. in a 5% CO2 atmosphere on an Olympus Fluo View FV3000 Confocal Laser Scanning Microscope, equipped with an Olympus 60× water objective (UPLSAPO60XW) and 405 and 640 nm lasers, unless otherwise stated. All samples were excited at 405 nm, and fluorescence emission collected from 510-610 nm. Images were processed using FIJI software. Data is presented as the mean fluorescence intensities of 40-50 cell clusters chosen as regions of interest from 10 images representative of each condition.

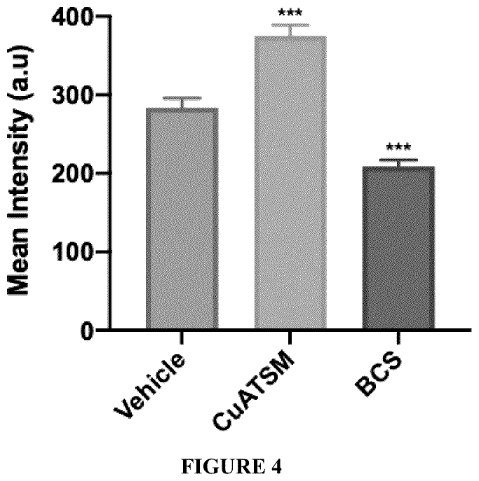

FIG. 4 shows mean fluorescence intensities of F—NpCul (10 μM) in SH-SY5Y cells treated with a vehicle control, 10 μM CuATSM (copper-loading agent) or 100 μM BCS (copper-depleting agent) for 1 h. Mean intensity of 40-50 regions of interest from at least 10 images. *** P<0.001 v vehicle-treated cells, which shows that the F—NpCul probe is sensitive to both an accumulation and a deficiency of copper in these cells.

### DIVE Microscopy

In vivo experiments were conducted to demonstrate the detectability of the probe and associated metal levels in a mouse model.

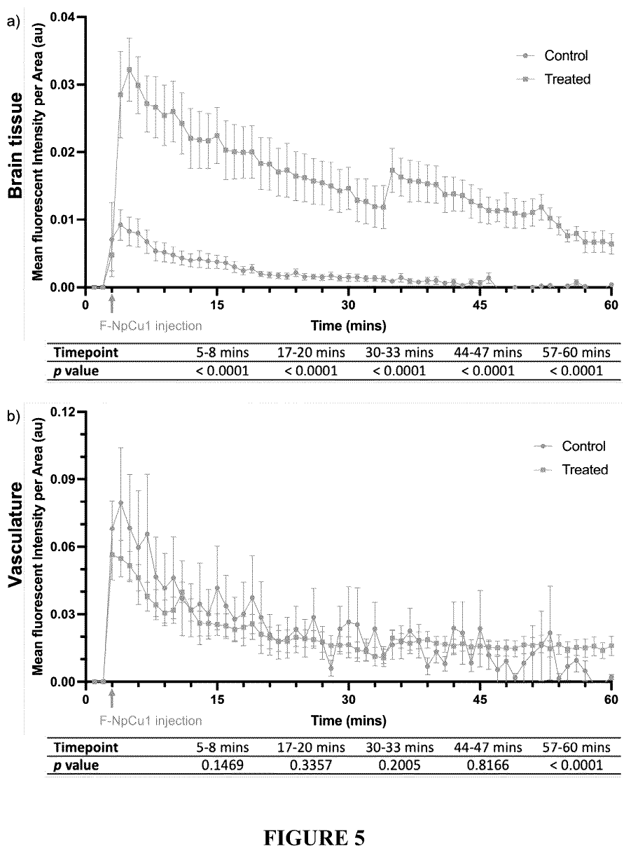

FIG. 5 shows fluorescentmission quantified longitudinally from real-time fluorescent images of anaesthetised SWISS mice obtained within the Deep In vivo Explorer (DIVE) microscope following F—NpCul injection. In this work, anaesthetised SWISS mice underwent a craniotomy surgery to expose a region of parietal cortex. Imaging was performed through this cranial window with a water dipping objective lens. 4 mg/kg F—NpCul was i.v. injected through the tail vein after three minutes. F—NpCul was excited at 920 nm and emission was 504-577nm. “Treated” mice (n=3) were orally administered the copper-delivery compound CuATSM (15 mg/kg) daily for eight days to increase brain copper levels prior to imaging while control mice (n=3, “Untreated”) received no treatment. For each mouse at all time points, fluorescent emission per unit area (arbitrary units) from five representative regions of interest (ROIs) within vasculature and five ROIs in surrounding tissue was quantified from a projected image combining 130 μm depth. Baseline fluorescence from an image acquired prior to probe injection was subtracted from all fluorescent measurements which were then averaged at each time point. Comparison of average fluorescent emission intensity between control and CuATSM-treated mice was undertaken for a) brain tissue and b) vasculature. Unpaired t-tests with significance p<0.05 were performed to describe the temporal pattern of change. Error bars show mean ±SEM.

In summary, data from FIG. 5 demonstrates the fluorescent signal from mice with higher tissue copper levels is significantly higher than that of mice with physiological brain tissue copper levels. Reported copper levels within the vasculature of treated vs untreated mice were equivalent.

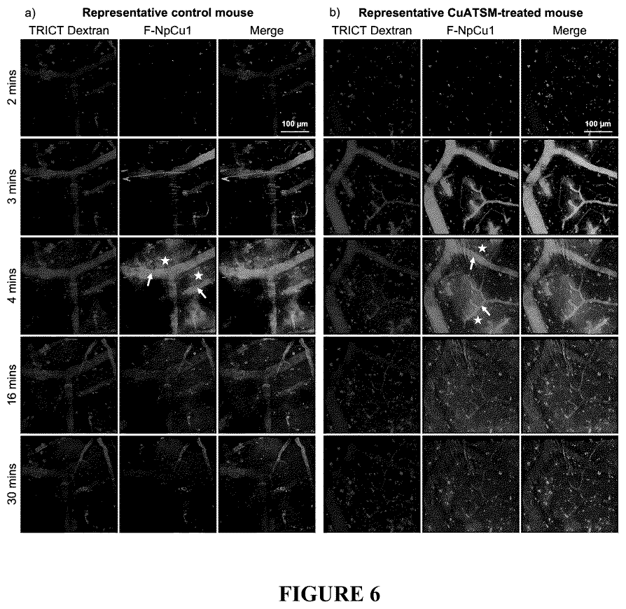

FIG. 6 shows representative fluorescent images from one control (untreated) and one CuATSM-treated (copper treatment) anaesthetised mouse injected with 4 mg/kg F—NpCul and imaged with the DIVE microscope. Higher fluorescent signal is observed in CuATSM-treated mice compared to control. Anaesthetised SWISS mice underwent a craniotomy surgery to create a cranial window exposing parietal cortex. Imaging was performed with a water dipping objective lens following tail vein i.v. injection of Dextran to visualise vasculature (red). Three minutes after imaging commenced, 4 mg/kg F—NpCul (green) was i.v. injected through the tail vein, and imaging continued for up to 60-minutes. Dextran and F—NpCul were excited at 920 nm, with 616-684 nm and 504-577 nm emission respectively. Each image in FIG. 6 is 1024×1024 pixels and shows a combined depth projection of 130 μm. Scale bar showing 100 μm applies to all images. In particular, FIG. 6a shows Control mouse received no treatment and thus represents physiological brain copper levels; FIG. 6b shows mouse treated with 15 mg/kg CuATSM orally/day for eight days. Arrows in FIG. 6 point to blood vessels and stars indicate signal surrounding vasculature.

In each of these in vivo studies, no signs of toxicity were noticed in the mice following F—NpCul exposure.

Although the invention has been described with reference to specific examples, it will be appreciated by those skilled in the art that the invention may be embodied in many other forms in particular features of any one of the various described examples may be provided in any combination in any of the other described examples. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

1. A probe for detecting a transition metal, the probe comprising:
a metal chelating portion of general formula I: 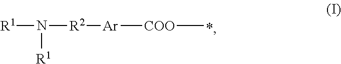
wherein:
Ar is an aromatic heterocycle comprising at least one nitrogen atom,
Each R¹ is independently an organic group of general formula R³—S—R^{3′}, R³—O—R^{3′}, or R³—N—R³,
Each of R², R³, and R^{3′} is independently a straight chained or branched C₁-C₆ alkyl, and
* is bound to the linker;
a label portion configured to be detectable, wherein the label portion comprises a radiolable; and
a linker bound to both the metal chelating portion and the label portion, wherein the linker is configured to react with a nucleophile when the metal chelating portion coordinates with the transition metal.

2. The probe of claim 1, wherein the transition metal is selected from Cu(I), Cu(II), Zn(II), Fe(II), Fe(III), Co(II), Mn(II), Ni(II) or Cd(II).

3. The probe of claim 1, wherein the metal chelating portion further comprises at least one nitrogen and/or sulfur and/or oxygen heteroatom which coordinates to the transition metal.

4. (canceled)

5. (canceled)

6. The probe of claim 1, wherein Ar is selected from the group consisting of pyrrolinyl, pyrrolyl, pyrazolinyl, imidazolinyl, imidazolyl, triazolyl, tetrazolyl, isothiazolyl, thiazolyl, thiadiazolyl, pyridinyl, pyridazinyl, pyrimidinyl, pyrazinyl, triazinyl and thiazinyl.

7. The probe of claim 1, wherein the metal chelating portion is

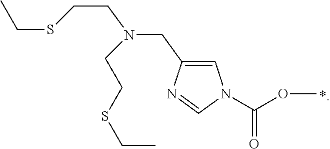 [SMILES: CCSCCN(CCSCC)Cc1cn(C(=O)OC)cn1]

8. (canceled)

9. (canceled)

10. The probe of claim 1, wherein the label portion comprises a naphthalimide fluorophore.

11. (canceled)

12. The probe of claim 10, wherein the fluorophore is 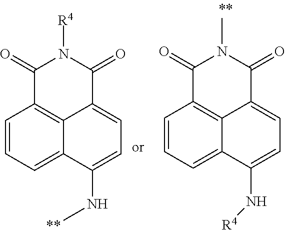
wherein:
R⁴ is C₁-C₆alkyl, optionally substituted with one or more substituents selected from amino, carboxyl, C₃-C₆ aryl and halo, wherein the aryl may be further substituted with one or more substituents selected from amino, carboxyl, C₁-C₆alkyl, C₃-C₆ aryl and halo and optionally comprising 1, 2, or 3 heteroatoms each selected from a nitrogen atom, an oxygen atom and a sulfur atom; and
** is bound to the linker.

13. (canceled)

14. The probe of claim 1, wherein the radiolabel is selected from the group consisting of ¹¹C, ¹³N, ¹⁵O, ¹⁸F and ¹³¹I.

15. The probe of claim 1, wherein the linker is C₁-C₆alkyl, optionally comprising 1, 2 or 3 heteroatoms selected from a nitrogen atom, an oxygen atom and a sulfur atom or any combination thereof and is covalently bound to both the metal chelating portion and the label portion.

16. (canceled)

17. (canceled)

18. The probe of claim 1, which is a compound selected from: 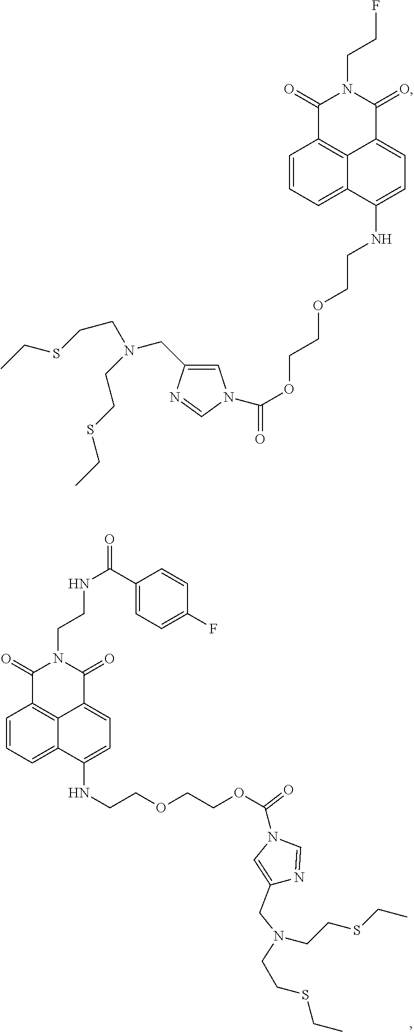 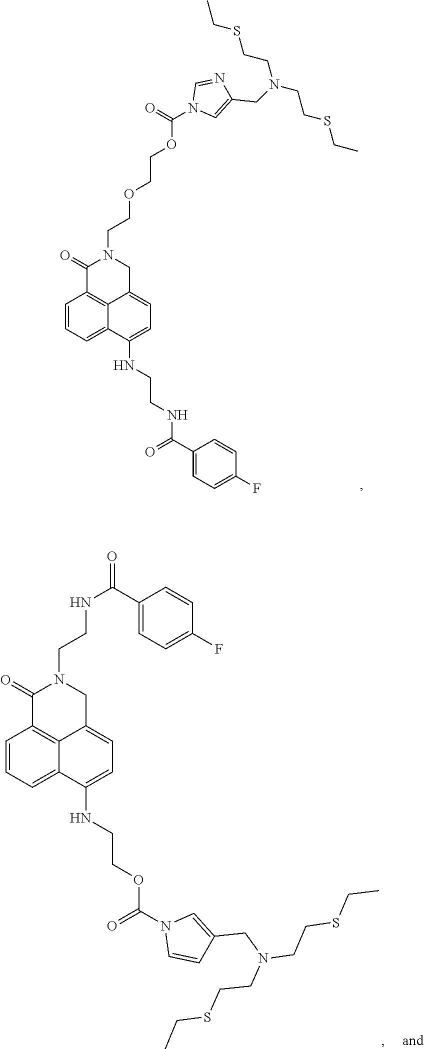 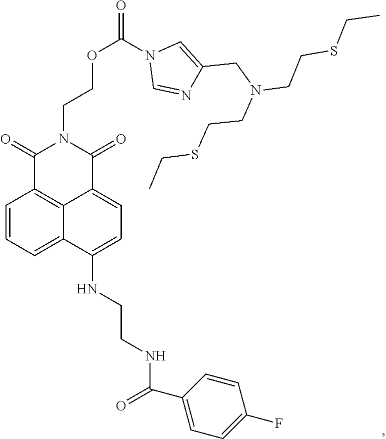
wherein F is ¹⁸F or ¹⁹F.

19. The probe of claim 18, having the structure: 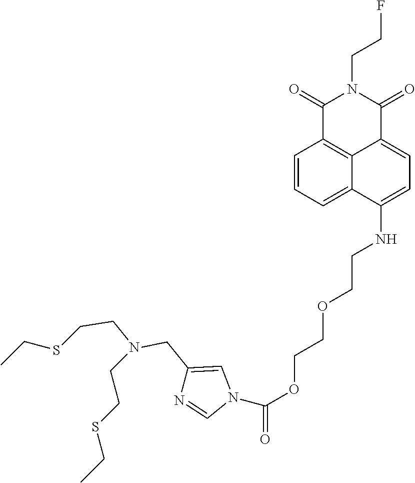
wherein F is ¹⁸F or ¹⁹F.

20. (canceled)

21. (canceled)

22. A method for measuring a transition metal in vivo, comprising administering to a subject an effective amount of the probe of claim 1 and detecting the label portion after a period of time between administering the probe to the subject and detecting the label portion, wherein the period of time is between about 3 minutes and about 1 hour, wherein the measuring comprises detecting the label portion, and/or locating regions of high or low intensity of the label portion, and wherein the administering is selected from oral administration, subcutaneous injection or infusion, and intravenous injection or infusion.

23. (canceled)

24. The method of claim 22, wherein measuring the transition metal in vivo is used for identifying a region of transition metal dyshomeostasis in the subject, the method further comprising comparing regions of high or low intensity of the label portion to a control subject without transition metal dyshomeostasis.

25. The method of claim 24, wherein the region of transition metal dyshomeostasis in the subject comprises a region of the central nervous system.

26. (canceled)

27. The method of claim 25, wherein the region of transition metal dyshomeostasis in the central nervous system of the subject comprises the brain and is indicative of a neurological condition selected from stroke, Parkinson's disease, Alzheimer's disease, amyotrophic lateral sclerosis (ALS), Wilson's disease, multiple sclerosis (MS), Menkes disease and neuroblastoma.

28. The method of claim 22, wherein measuring the transition metal in vivo is used for diagnosing a condition in a subject, further comprising the steps of:
forming a subject map of label intensity by locating regions of high or low intensity of the label portion in at least one biological system of the subject;
and
comparing the subject map of label intensity to a control map of label intensity;
whereby a region of relative low intensity or relative high intensity in the subject map of label intensity is indicative of a condition.

29. The method of claim 28, wherein the at least one biological system of the subject comprises the central nervous system, the hepatic system, the endocrine system, or any combination thereof, and the condition is selected from Parkinson's disease, Alzheimer's disease, stroke, amyotrophic lateral sclerosis (ALS), Wilson's disease, multiple sclerosis (MS), Menkes disease, copper storage hepatopathy, diabetes mellitus, and neuroblastoma.

30-35. (canceled)

36. A method of labelling a site of a transition metal in a biological tissue, comprising:
(a) contacting the probe of claim 1 with the biological tissue, wherein the metal chelating portion of the probe binds to the transition metal, whereby binding of the transition metal to the metal chelating portion cleaves the metal chelating portion from the linker to form a reactive acyl group;
(b) contacting the reactive acyl group with a nucleophile, resulting in binding of the label portion and the linker to the nucleophile; and
(c) measuring the location of the label portion after a period of time between about 3 minutes and about 1 hour after contacting the probe with the biological tissue.

37. The method of claim 36, wherein the nucleophile is an amino acid.

38. The probe of claim 10, wherein the label is detectable by both fluorescence and PET imaging.
